(19) United States
(12) Patent Application Publication
(10) Pub. No.: US 2019/0133125 A1
(43) Pub. Date: May 9, 2019

(54) ANTIMICROBIAL POLYMER

(71) Applicant: BASF SE, Ludwigshafen Am Rhein (DE)

(72) Inventors: Jürgen WIETHAN, Emmendingen (DE); Jean-Pierre Berkan LINDNER, Mannheim (DE)

(21) Appl. No.: 15/751,335

(22) PCT Filed: Aug. 4, 2016
(86) PCT No.: PCT/EP2016/068660

§ 371 Date: Feb. 8, 2018

(30) Foreign Application Priority Data
Aug. 11, 2015 (EP) 15180551.2
Oct. 9, 2015 (EP) 15189053.0
Oct. 29, 2015 (EP) 15192124.4
Apr. 18, 2016 (EP) 16165774.7

(51) Int. Cl. A01N 43/50; C08G 73/06; A61K 31/787; A61K 8/84; A61Q 15/00; A61K 45/06; C11D 3/48

(52) U.S. Cl. CPC A01N 43/50; C08G 73/0616; A61K 31/787; C11D 3/48; A61Q 15/00; A61K 45/06; A61K 8/84

(57) ABSTRACT

Disclosed is a polymeric, ionic compound comprising at least 5 imidazolium groups, which compound is obtainable by simultaneously reacting glyoxal, formaldehyde, at least one C₄-C₈alkylene diamine, at least one protic acid, and a C₈-C₂₀alkylamine, especially by simultaneously reacting glyoxal, formaldehyde hexylene diamine, acetic acid and dodecylamine. The polymeric, ionic compound comprising at least 5 imidazolium groups shows advantageous antimicrobial properties, and may be used in various formulations for application fields including plant protection, personal care, home care, industrial or institutional or hospital disinfection, material protection, or pharmaceuticals.

The present invention relates to a new formulation with antimicrobial activity, to a new antimicrobial polymer, to the use of said formulation and said polymer as a biocide e.g. in formulations for cleaning, disinfection and/or descaling purposes, as well as to a cleaning agent or disinfection agent or descaling agent comprising said formulation.

WO 2012/127009 describes a number of imidazolium polymers with activity against Staphylococcus aureus and Pseudomonas aeruginosa. It has now been found that certain polymers of this class may be derivatized inter alia to obtain defined end groups, and that such modified polymers unexpectedly show an improved antimicrobial action, It has now been found that a certain class of such linear imidazolium polymers shows a distinctly improved biocidal effect when derivatized to obtain alkyl end groups (“alkyl capping”), especially when certain molecular weight ranges are met.

The invention thus provides polymeric, ionic compound comprising at least 5 imidazolium groups, typically at least 8 imidazolium groups, which contains alkyl moieties as end groups, which compound shows a good antimicrobial activity. In the following, the compound of the invention will be referred to as a polymeric compound, polyimidazolium or imidazolium polymer, notwithstanding the fact that properties of valuable species of short chain length (i.e. compounds comprising 8, 7, 6 or even only 5 imidazolium moieties in total) may vary with chain length, which species thus could, alternatively, be described as oligomers.

The alkyl end groups are typically selected from the group consisting of C₈-C₂₀alkyl groups, which may be straight chain or branched, such as octyl, 2-ethylhexyl, nonyl, decyl, undecyl, dodecyl, tridecyl, tetradecyl, pentadecyl, hexadecyl, heptadecyl, octadecyl, eicosyl.

Present end-capped imidazolium polymer is advantageously prepared in analogy to methods described in WO 2012/127009, WO 2010/072571, and literature cited therein by reaction of glyoxal (i.e. ethane-1,2-dione), formaldehyde and alkylene diamine, where, in addition, a defined end group is introduced. This can be effected e.g. by reacting the product obtained in WO 2012/127009 with a suitable alkylating agent such as an alkyl halogenide. Generally, the polymer of the invention is obtainable by one of the methods (i) to (iii):

Method (i) comprises reacting glyoxal, formaldehyde, at least one C₄-C₈alkylene diamine, at least one protic acid, and a C₈-C₂₀alkylamine.

Method (ii) comprises a first step of reacting glyoxal, formaldehyde, at least one C₄-C₈alkylene diamine and at least one protic acid, followed by a second step of reacting the product obtained in the first step with a suitable C₈-C₂₀alkyl compound such as a C₈-C₂₀alkyl halogenide.

Method (iii) comprises a first step of reacting glyoxal, formaldehyde, at least one C₄-C₈alkylene diamine, at least one protic acid and a C₈-C₂₀alkylamine, followed by a second step of reacting the product obtained in the first step with a suitable C₈-C₂₀alkyl compound such as a C₈-C₂₀alkyl halogenide.

According to a preferred method, endcapping is effected by adding certain amounts of monoamine during the polymerization reaction. Imidazolium polymers carrying a defined end group different from the used diamine are thus obtainable by adding a mono-amine, which carries the desired functional group, to the reaction mixture. The amount of mono-amine often ranges from about 1 to 25 mol % of the amount of diamine, preferably from about 8 to 20 mol %, for example the amount of monoamine is about 10 mol % of the diamine. In a typical synthesis, especially according to method (i), 1 molar part of aldehyde, about 0.9-1.1 molar parts of the alpha-dicarbonyl compound, about 0.9-1.0 molar parts of the diamine, 0.01-0.2 molar parts of the monoamine, and an excess (typically about 1.1 to 5 molar parts) of the protic acid. Where low amounts of the monoamine are employed (method iii) or, other than described for the above typical synthesis, no monoamine is added, the polymer obtained may be reacted in a second step with a suitable alkylating agent as described above for methods (ii) and (iii). In the polymer obtained, the imidazolium groups are joined to one another by the alkylene groups resulting from the diamine educt, and the polymer is terminated by the mono-amine. In one embodiment of the invention, a polymer with fully alkylated end groups is obtained. It has been found, however, that a product comprising a certain amount of monoalkylated species (i.e. embodiment 2), including mixtures of polymer species having 2 alkyl end groups, polymers having only one alkyl end group (i.e. monoalkylated species), and even a certain amount of non-alkylated species, also may show the desired improvement. The total degree of alkylation (percentage of alkyl end groups on all end groups of the present linear imidazolium polymers) thus may be about 100% (embodiment 1), for example 95-100%. The total degree of alkylation (percentage of alkyl end groups on all end groups of the present linear imidazolium polymers) thus may alternatively be from the range 50 to 100% (embodiment 2), for example 50-95%, especially 60 to 90%.

Protic acids are, for example, selected from C₁-C₂₀carboxylic acids and mineralic acids; such as formic acid, hydrohalogenides like hydrochloric acid and hydrobromic acid, acetic acid, aliphatic carboxylic acids having 3 to 20 carbon atoms, H₂SO₄, HNO₃, H3PO4, C₁-C₈sulfonic acids, trifluormethyl-sulfonic acid; especially preferred is acetic acid.

For the present polymers, formaldehyde (HCHO) is used as the aldehyde, and glyoxal (ethanedial, formula OCHCHO) is used as the alpha-dicarbonyl compound.

The diamine used for preparing the present polymers is a C₄-C₈alkylene diamine, typically containing a straight chain alkylene group carrying terminated by NH₂ groups. Preferred is n-hexylene-1,6-diamine (i.e. 1,6-diaminohexane).

The monoamine is selected from (preferably straight chain) C₈-C₂₀alkylamines, whose alkyl groups are as defined above; preferred is dodecylamine.

The polymer obtained contains imidazolium groups, whose charge is generally balanced by counterions formed by carboxylic or mineralic acids. Preferred are carboxylic acids, e.g. selected from formic acid, acetic acid, propanoic acid, oxalic acid, lactic acid, citric acid, tartaric, mandelic acid, benzoic acid, salicylic acid, glutaric acid, sorbic acid and succinic acid; more preferred are aliphatic carboxylic acids like formic acid, acetic acid, propanoic acid, and long chain fatty acids, especially formic acid, acetic acid, propanoic acid, for use as counterions. Furthermore mineralic acids like H2SO4, HNO3, H3PO4, sulfonic acids like p-toluene sulfonic acid, methanesulfonic acid, trifluormethyl-sulfonic acid may be used.

The imidazolium polymer thus obtained may be subjected to an anion exchange. This allows the preparation of imidazolium polymers even with anions for which no corresponding stable protic acid exists. The anion exchange can be effected by known methods, e.g. transprotonation, reaction with a metal salt, ion exchange chromatography, electrolytically or by means of a combination of these measures. Anions of the present polymer (expressed as A_{C}⁻ in the below formula I) thus may comprise any convenient anion, such as halide, sulfate, hydrogensulfate, alkylsulfate, arylsulfate, perfluoro alkyl- and arylsulfates, sulfonate, alkylsulfonate, arylsulfonate, perfluoro alkyl- and arylsulfonates, phosphate, halophosphate, alkylphosphate, nitrate, perchlorate, tetrachloroaluminate, tetrafluoroborate, alkylborate, saccharinate, alkyl carboxylates or bis(perflouroalkylsulfonyl)anions. Anions also comprise halogenides and pseudohalogenides such as F⁻, Cl⁻, Br⁻, I⁻, CN⁻, SCN⁻, OCN⁻, NO₂⁻, NO₃⁻, N(CN)⁻, or BF₄⁻, PF₆⁻, AlF₄⁻, AlCl₄⁻, AlBr₄⁻, ZnCl₃⁻, SnCl₄⁻, CuCl₂⁻; the group of sulfates, sulfites and sulfonates, the group of phosphates; the group of phosphonates and phosphinates; the group of phosphites; the group of phosphonites and phosphinites; the group of carboxylates and polybasic carboxylic acids; the group of boronates; the group of silicates and silicic esters; the group of alkylsilane and arylsilane salts; the group of carboximides, bis(sulfonyl)imides and sulfonylimides. A typical polymer thus obtained is of the formula I

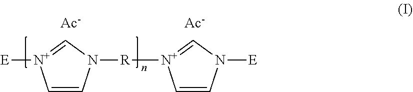

wherein

E is C₈-C₂₀ alkyl, preferably of straight chain, and especially is dodecyl;

R is C₄-C₈alkylene, preferably of straight chain, and especially is n-hexylene;

A_{C}⁻ is the anion of the protonic acid, typically of a carboxylic or mineralic acid as described above, or an anion obtained by ion exchange as described above, and especially is an acetyl anion; and

the index n ranges from 4 to 10000, preferably from 5 to 1000, especially from 6 to 100.

Of specific technical importance are polymeric ionic compounds obtainable according to the methods described above and comprising at least 8 imidazolium groups, such as compounds of the formula I wherein n ranges from 7 to 1000, including products wherein n ranges from 7 to 100. Some preferred compounds of the formula I are those wherein n ranges from 5 to 50, or wherein n ranges from 7 to 50.

Since partial alkylation of the end-caps also leads to the desired result, a typical polymer obtained in the present invention may alternatively be of the formula II

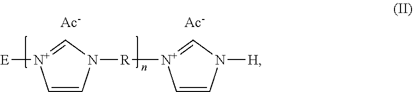

wherein symbols are as defined above for formula I. It is to be noted that species of the above formula II may exist in tautomeric forms, exemplified by the below formula II′

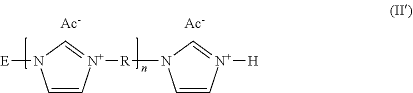

which are understood to fall under formula II as well as under below formula I′, for the purpose of the present specification.

Thus, the polymer of the invention generally is of formula I′

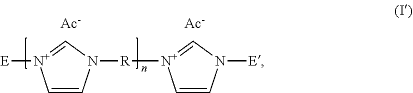

E is C₈-C₂₀alkyl, preferably of straight chain, and especially is dodecyl;

E′ is as defined for E, or is hydrogen, and all other symbols are as defined above for formula I.

The molecular weight of the antimicrobial polymer of the invention typically ranges from 3000 to 100000, especially 5000 to 25000, with respect to the weight average Mw, and from 800 to 15000, especially 1000 to 2500, with respect to the number average Mn. Preferred is an antimicrobial polymer of the invention showing a polydispersity (Mw/Mn) from the range 5 to 25, especially from the range 5 to 10. The invention thus includes products comprising varying chain length, such as polymeric ionic compounds obtainable as described above and characterized by a molecular weight as noted hereinabove and showing a polydispersity (Mw/Mn) from the range 5 to 25, especially from the range 5 to 10, including a composition comprising more than one individual compound of the formula I, wherein the compounds containing imidazolium units within their polymer chain show a polydispersity (Mw/Mn) from the range 5 to 25, especially from the range 5 to 10.

The invention thus includes a process for the preparation of a polymeric, ionic compound comprising at least 5 imidazolium groups, including a polymer of the formula I as described above, which process comprises simultaneously reacting glyoxal, formaldehyde, at least one C₄-C₈alkylene diamine, at least one protic acid, and a C₈-C₂₀alkylamine, especially by simultaneously reacting glyoxal, formaldehyde, hexylene diamine, acetic acid and dodecylamine. Preferably, 0.9-1.1 molar parts of glyoxal are reacted with 1 molar part of formaldehyde, 0.9-1.0 molar parts of the alkylene diamine, 0.01-0.2 molar parts of the alkylamine, and about 1.1 to 5 molar parts of the protic acid.

The reaction is typically carried out by mixing the reactands, e.g. by adding the reactands to the protic acid, in the presence of a suitable solvent, e.g. a polar solvent such as water or mixtures of water with other protic solvents such as an alcohol. Temperature of the mixture is preferably controlled during this step to remain in the lower liquid range, such as −20° C. to about 40° C., or 0 to 30° C. Pressure is only of minor importance, e.g. to ensure a certain liquid range such as about −5° to about 100° C. in case of atmospheric pressure and use of water as the solvent. After addition of all components, the reaction mixture advantageously is heated e.g. to about 60 to 150° C., or up to the boiling point, for a time period of about 0.1 to 10 hours. After completion of the reaction, the mixture comprising the polymer of the invention typically has a pH from the range 3-7, e.g. 5-6 in case that acetic acid is used as the protic acid, and may be used as such, or may be subjected to further derivatization (e.g. reacting with alkylating agent) and/or purification steps (e.g. removal of the solvent under reduced pressure and/or heating to the boiling point).

Formulations

The invention thus primarily pertains to an antimicrobial composition comprising, as an antimicrobial agent, at least one polymeric, ionic compound comprising at least 5, in preferred products at least 6 or 7 or especially at least 8 imidazolium groups, and at least one carrier and/or at least one auxiliary agent, wherein the polymeric compound is obtainable by one of the methods (i) to (iii).

The antimicrobial composition according to the invention can be provided and/or applied as a solid or as a liquid. This encompasses compositions in form of aerosols. The biocide composition according to the invention can be formulated e.g. as powder, granulate, pellets, pills, agglomerates, solutions, emulsions, suspensions, dispersions, pastes, in combination with carrier materials, etc.

The antimicrobial compositions according to the invention can be formulated free from solvent or with a suitable solvent as a carrier. Generally, the imidazolium compounds used according to the invention are soluble in most protic solvents, swellable in most aprotic polar solvents and insoluble in most nonpolar solvents. Solvents suitable as carriers for the biocide compositions according to the invention are selected from among water, alcohols, such as methanol, ethanol, n-propanol, isopropanol, n-butanol, tertbutanol, diols and polyols, such as ethanediol and propanediol, amino alcohols, such as ethanolamine, diethanolamine and triethanolamine, ethers, e.g. tetrahydrofuran, diethyl ether, methyl tert-butyl ether and diethylene glycol monomethyl ether, ketones, such as acetone and methyl ethyl ketone, esters, e.g. ethyl acetate, formamide, dimethylformamide (DMF), dimethylacetamide, dimethyl sulfoxide (DMSO), acetonitrile, aromatic solvents, e.g. benzene, toluene, ethylbenzene or xylenes, halogenated solvents, e.g. dichloromethane, chloroform, carbon tetrachloride, dichloroethane or chlorobenzene, aliphatic solvents, e.g. pentane, hexane, heptane, octane, ligroin, petroleum ether, cyclohexane and decalin, and mixtures thereof. The solvent is preferably selected from among water, water-miscible organic solvents and mixtures thereof. The solvent is particularly preferably selected from among water, methanol, ethanol, n-propanol, isopropanol, n-butanol, tert-butanol and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

A multitude of different auxiliaries such as active substances and additives can be formulated in the biocide compositions according to the invention. Suitable auxiliaries include surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, antifoaming agents, colorants, tackifiers and binders.

The carrier and/or auxiliary agent preferably comprises a solvent, such as water and/or one or more alcohols, and a surfactant.

Suitable surfactants are surface-active compounds, such as cationic, nonionic and amphoteric surfactants, block polymers, polyelectrolytes, and mixtures thereof. In certain cases, anionic surfactants may be applied as well, e.g. in combination with nonionic or amphoteric surfactants. Such surfactants can be used as emulsifier, dispersant, solubilizer, wetter, penetration enhancer, protective colloid, or adjuvant. They are for instance described in the disclosure IP.com Journal (2011), 12(1A), IPCOM000213522D, published on 20 Dec. 2011 in section (b). Examples of surfactants are listed in McCutcheon's, Vol. 1: Emulsifiers & Detergents, McCutcheon's Directories, Glen Rock, USA, 2008 (International Ed. or North American Ed.).

Suitable adjuvants are compounds which have a neglectable or even no pesticidal activity themselves, and which improve the biological performance of the polymeric, ionic compound comprising imidazolium groups on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellu-lose), anorganic clays (organically modified or unmodified), polycarboxylates, and silicates. Suitable bactericides and antimicrobial agents, such as bronopol and isothiazolinone derivatives, are as listed below.

Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers

For example, the composition of the invention may comprise auxiliaries, such as 0.1-1 wt % further antimicrobial agents, 5-15 wt % anti-freezing agents, 0.1-1 wt % anti-foaming agents, and 0.1-1 wt % colorants.

Cationic, non-ionic and anionic surfactants are widely known in the art.

Suitable cationic surfactants include quaternary surfactants, for example quaternary ammonium compounds with one or two hydrophobic groups, or salts of long-chain primary amines. Preference is given to ammonium halides, such as alkyltrimethylammonium chlorides, dialkyldimethylammonium chlorides and trialkylmethylammonium chlorides, for example cetyltrimethylammonium chloride, stearyltrimethylammonium chloride, distearyldimethy-lammonium chloride, lauryldimethylammonium chloride, lauryldimethylbenzylammonium chloride and tricetylmethylammonium chloride. Further cationic surfactants that can be used in accordance with the invention are quaternised protein hydrolysates.

Also suitable in accordance with the invention are cationic silicone oils, such as, for example, the commercially available products Q2-7224 (manufacturer: Dow Corning; a stabilised trimethylsilylamodimethicone), Dow Corning 929 emulsion (comprising a hydroxylamino-modified silicone, which is also referred to as amodimethicone), SM-2059 (manufacturer: General Electric), SLM-55067 (manufacturer. Wacker) and also Abil®-Quat 3270 and 3272 (manufacturer: Th. Goldschmidt; diquaternary polydimethylsiloxanes, quaternium-80), or silicones, as described in WO 00/12057, especially page 45, line 9 to page 55, line 2.

Alkylamidoamines, especially fatty acid amidoamines, such as the stearylamidopropyldimethylamine obtainable under the name Tego Amid® 18, are distinguished not only by a good conditioning action but also especially by their good biodegradability. Quaternary ester compounds, so-called “esterquats”, such as the methyl hydroxyalkyl-dialkoyloxyalkylammonium methosulfates marketed under the trademark Stepantex®, are also very readily biodegradable.

An example of a quaternary sugar derivative that can be used as cationic surfactant is the commercial product Glucquat®100, according to CTFA nomenclature a “lauryl methyl gluceth-10 hydroxypropyl dimonium chloride”.

A wide variety of anionic surfactants are potentially useful herein. Several examples of suitable anionic surfactants are disclosed in U.S. Pat. No. 3,929,678, which is incorporated herein by reference.

Examples of anionic surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, and mixtures thereof. Mixtures of anionic surfactants can be used effectively in the present invention.

Alkyl and alkyl ether sulfates have the respective formulae R₁—O—SO₃-M and R₁—(CH₂H₄—O)ₓ—O—SO₃-M, wherein R₁ is a saturated or unsaturated, branched or unbranched alkyl group from about 8 to about 24 carbon atoms, x is 1 to 10, and M is a water-soluble cation such as ammonium, sodium, potassium, magnesium, triethanolamine, diethanolamine and monoethanolamine. The alkyl sulfates are typically made by the sulfation of monohydric alcohols (having from about 8 to about 24 carbon atoms) using sulfur trioxide or other known sulfation technique. The alkyl ether sulfates are typically made as condensation products of ethylene oxide and monohydric alcohols (having from about 8 to about 24 carbon atoms) and then sulfated. These alcohols can be derived from fats, e.g., coconut oil or tallow, or can be synthetic. Common examples of alkyl sulfates include sodium, ammonium, potassium, magnesium, or TEA salts of lauryl or myristyl sulfate. Common examples alkyl ether sulfates include ammonium, sodium, magnesium, or TEA laureth-3 sulfate.

Another class of anionic surfactants potentially useful are sulfated monoglycerides of the formula R₁—CO—O—CH₂—C(OH)H—CH₂—O—SO₃-M, wherein R₁ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are typically made by the reaction of glycerin with fatty acids (having from about 8 to about 24 carbon atoms) to form a monoglyceride and the subsequent sulfation of this monoglyceride with sulfur trioxide. An example of a sulfated monoglyceride is sodium cocomonoglyceride sulfate.

Other anionic surfactants potentially useful include olefin sulfonates of the form R₁SO₃-M, wherein R₁ is a mono-olefin having from about 12 to about 24 carbon atoms, and M is a water-soluble cation such as ammonium, sodium, potassium, magnesium, triethanolamine, diethanolamine and monoethanolamine. These compounds can be produced by the sulfonation of α-olefins by means of uncomplexed sulfur trioxide, followed by neutralization of the acid reaction mixture in conditions such that any sultones which have been formed in the reaction are hydrolyzed to give the corresponding hydroxyalkanesulfonate. An example of a sulfonated olefin is sodium C₁₄/C₁₆ α-olefin sulfonate.

Other anionic surfactants potentially useful are the linear alkylbenzene sulfonates of the form R₁—C₆H₄—SO₃-M, wherein R₁ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are formed by the sulfonation of linear alkyl benzene with sulfur trioxide. An example of this anionic surfactant is sodium dodecylbenzene sulfonate.

Still other anionic surfactants include the primary or secondary alkane sulfonates of the form R₁—SO₃-M, wherein R₁ is a saturated or unsaturated, branched or unbranched alkyl chain from about 8 to about 24 carbon atoms, and M is a water-soluble cation such as ammonium, sodium, potassium, magnesium, triethanolamine, diethanolamine and monoethanolamine. These are commonly formed by the sulfonation of paraffins using sulfur dioxide in the presence of chlorine and ultraviolet light or another known sulfonation method. The sulfonation can occur in either the secondary or primary positions of the alkyl chain. An example of an alkane sulfonate useful herein is alkali metal or ammonium C₁₃-C₁₇ paraffin sulfonates.

Still other anionic surfactants potentially useful are the alkyl sulfosuccinates, which include disodium N-octadecylsulfosuccinamate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Also useful may be taurates which are based on taurine, also known as 2-aminoethanesulfonic acid. Examples of taurates include N-alkyltaurines such as the one prepared by reacting dodecylamine with sodium isethionate according to the teaching of U.S. Pat. No. 2,658,072 which is incorporated herein by reference in its entirety. Other examples based of taurine include the acyl taurines formed by the reaction of n-methyl taurine with fatty acids (having from about 8 to about 24 carbon atoms).

Another class of anionic surfactants potentially useful are the acyl isethionates. The acyl isethionates typically have the formula R₁—CO—O—CH₂—CH₂SO₃-M, wherein R₁ is a saturated or unsaturated, branched or unbranched alkyl group having from about 10 to about carbon atoms, and M is a cation. These are typically formed by the reaction of fatty acids (having from about 8 to about 30 carbon atoms) with an alkali metal isethionate. Non-limiting examples of these acyl isethionates include ammonium cocoyl isethionate, sodium cocoyl isethionate, sodium lauroyl isethionate, and mixtures thereof.

Still other anionic surfactants potentially useful are the alkylglyceryl ether sulfonates of the form R₁—OCH₂—C(OH)H—CH₂—SO₃-M, wherein R₁ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These can be formed by the reaction of epichlorohydrin and sodium bisulfite with fatty alcohols (having from about 8 to about 24 carbon atoms) or other known methods. One example is sodium cocoglyceryl ether sulfonate.

Other anionic surfactants potentially useful include the sulfonated fatty acids of the form R₁—CH(SO₄)—COOH and sulfonated methyl esters of the from R₁—CH(SO₄)—CO—O—CH₃, where R₁ is a saturated or unsaturated, branched or unbranched alkyl group from about 8 to about 24 carbon atoms. These can be formed by the sulfonation of fatty acids or alkyl methyl esters (having from about 8 to about 24 carbon atoms) with sulphur trioxide or by another known sulfonation technique. Examples include alpha sulphonated coconut fatty acid and lauryl methyl ester.

Other anionic materials potentially useful as surfactants include alkyl ether carboxylates corresponding to the formula R₁—(OCH₂CH₂)x-OCH₂—CO₂-M wherein R₁ is a saturated or unsaturated, branched or unbranched alkyl or alkenyl group of about 8 to about 24 carbon atoms, x is 1 to 10, and M is a water-soluble cation. Nonlimiting examples of which include sodium laureth carboxylate.

Other anionic materials potentially useful as surfactants include acyl lactylates corresponding to the formula R₁—CO—[O—CH(CH₃)—CO]ₓ—CO₂-M wherein R₁ is a saturated or unsaturated, branched or unbranched alkyl or alkenyl group of about 8 to about 24 carbon atoms, x is 3, and M is a water-soluble cation, non-limiting examples of which include sodium cocoyl lactylate.

Other anionic materials potentially useful as surfactants include the carboxylates, nonlimiting examples of which include sodium lauroyl carboxylate, sodium cocoyl carboxylate, and ammonium lauroyl carboxylate. Anionic flourosurfactants can also be used.

Other anionic materials include acyl glutamates corresponding to the formula R₁—CO—N(COOH)—CH₂CH₂—CO₂-M wherein R, is a saturated or unsaturated, branched or unbranched alkyl or alkenyl group of about 8 to about 24 carbon atoms, and M is a water-soluble cation. Nonlimiting examples of which include sodium lauroyl glutamate and sodium cocoyl glutamate.

Other anionic materials include alkanoyl sarcosinates corresponding to the formula R₁—CON(CH₃)—CH₂CH₂—CO₂-M wherein R₁ is a saturated or unsaturated, branched or unbranched alkyl or alkenyl group of about 10 to about 20 carbon atoms, and M is a water-soluble cation. Nonlimiting examples of which include sodium lauroyl sarcosinate, sodium cocoyl sarcosinate, and ammonium lauroyl sarcosinate.

Any counter cation, M, can be used on the anionic surfactant. Typically the counter cation is selected from the group consisting of sodium, potassium, ammonium, monoethanolamine, diethanolamine, and triethanolamine.

Typical non-ionic surfactants are condensated products of ethylene oxide with various reactive hydrogen-containing compounds reactive therewith having long hydrophobic chains (e.g. aliphatic chains of about 8 to 20 carbon atoms), which condensation products (“ethoxamers”) contain hydrophilic polyoxyethylene moieties, such as condensation products of poly(ethyleneoxide) with fatty acids, fatty alcohols, fatty amides, polyhydric alcohols (e.g. sorbitan monostearate) and polypropylene oxide. Polyoxamers are e.g. block copolymers of polyoxyethylene and polyoxypropylene having an average molecular weight from about 3000 to 5000 and a preferred average molecular weight from about 3500 to 4000 and containing about 10-80% hydrophilic polyoxyethylene groups, by weight, of the block copolymer.

An important class of non-ionic surfactants are the alkylated polyglycosides (also known as “sugar surfactant”) comprising a carbohydrate as hydrophilic moiety and fatty alcohols as hydrophobic component, which may be represented by the general formula:

R—O-Gₚ

where R is alkyl or alkenyl (especially C₈-C₁₄),
G is the residue of an aldose or ketose and
P is 1, 2, 3, 4, 5, 6, 7, 8, 9 or 10. Examples of such surfactants, and their combinations with antimicrobial agents, which may also be used within the present invention, are disclosed in WO 2013/045340.

In a further variant, the sugar surfactants to be used according to the invention are alkyl and/or alkenyl polyglycosides of the above formula in which R is an alkyl and/or alkenyl radical having 4 to 22 carbon atoms, G is a sugar radical having 5 or 6 carbon atoms and P is numbers from 1 to 10. In a further embodiment, the alkyl and/or alkenyl polyglycosides are derived from aldoses or ketoses having 5 or 6 carbon atoms.

The component G in the above formula is selected in one embodiment from the group of hexoses, preferably from the group comprising allose, altrose, glucose, mannose, gulose, idose, galactose, talose, psicose, fructose, sorbose, tagatose, particularly preferably glucose.

In a further embodiment, the component G in the above formula is selected from the group of pentoses, preferably the group comprising ribulose, xylulose, ribose, arabinose, xylose, lyxose, particularly preferably xylose and/or arabinose.

As amphoteric surfactants C₈-C₁₈-betains, C₈-C₁₈-sulfobetains, C₈-C₂₄-alkylamido-C₁-C₄-alkylene betaines, imidazoline carboxylates, alkylamphocarboxycarbonic acids, alkylamphocarbonic acid (e.g. lauroamphoglycinate) and N-alkyl-β-aminopropionate or -iminodipropionate can be used. In particular the C₁₀-C₂₀-alkylamidoC₁-C₄-alkylenbetaine and coco fatty acid amide propylbetaine.

Examples of non-ionic surfactants include the zwitterionic and amphoteric surfactants of U.S. Pat. Nos. 2,658,072, 2,438,091, and 2,528,378, which are incorporated herein by reference.

The composition of the invention contains the polymeric, ionic compound comprising at least 5 imidazolium groups typically in an amount ranging from 0.0001 to 30% by weight of the composition. Liquid concentrates (typically aqueous and or alcoholic, e.g. using a polyhydric alcohol such as propylene glycol as the solvent) may be formulated, which contain up to 70% by weight of the present imidazolium polymer, typically 10 to 70%; an example is an aqueous solution containing about 50% by weight of the polymer.

The formulation of the invention may comprise the polymeric imidazolium compound of the invention in the mixture as obtained, e.g. of pH from the range 5 to 6, or in a pH adjusted way (typically after addition of a suitable base such as NaOH or KOH, or after addition of a suitable acid such as HCl in case that a low pH is desired). Typical is an aqueous formulation of a pH from the neutral to acidic range, e.g. from the range of pH 3 to 7, e.g. 5 to 6, or an aqueous formulation, which has a pH from the range of 7 to 10; the aqueous formulation contains at least 50 wt.-%, especially 80 to 99.8 wt.-%, of water.

The formulation of the invention typically contains a surfactant, such as a cationic, an anionic or non-ionic surfactant selected; mixtures of anionic and non-ionic surfactants may also be used.

The imadazolium polymers used in accordance with the invention, as well as formulations of the invention containing such polymers, exhibit pronounced antimicrobial action, for example against pathogenic gram-positive bacteria, gram-negative bacteria, yeasts and moulds. They are effective against fungi and, most importantly, they retain their effectiveness against fungi even when in in combination with non-ionic surfactants. They further show pronounced anti-biofilm activity, preventing or inhibiting the formation of a bio-film, with disruption, kill and/or removal of an existing biofilm, which allows for the preparation of efficient formulations for clean-in-place (CIP), water treatment or sanitary disinfection, as described further below.

The formulation of the invention may contain further antimicrobial agents, such as
a chloro benzene derivative of the formula (II)

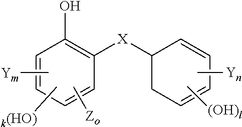

wherein

### X is O, S or —CH2-

### Y is Cl or Br,

Z is SO2H, NO2 or C1 to C4 alkyl,
k is 0 or 1,
l is 0 or 1,
m is 0, 1, 2, or 3,
n is 0, 1, 2, or 3,
o is 0 or 1,
which preferably is triclosan or diclosan (i.e. the compound 4,4′-dichloro-2-hydroxydiphenylether).

Other antimicromial agents suitable for combining with the present imidazolium polymer include (alternative names in brackets; numbers: Chemical Abstracts Registry):
(benzothiazol-2-ylthio)methyl thiocyanate (TCMTB) 21564-17-0
(benzyloxy)methanol 14548-60-8
(ethylenedioxy)dimethanol (Reaction products of ethylene glycol with paraformaldehyde (EGForm)) 3586-55-8
.alpha.,.alpha.′,.alpha.″-trimethyl-1,3,5-triazine-1,3,5(2H,4H,6H)-triethanol (HPT) 25254-50-6
1,2-benzisothiazol-3(2H)-one (BIT) 2634-33-5
1,3-bis(hydroxymethyl)-5,5-dimethylimidazolidine-2,4-dione (DMDMH) 6440-58-0
1-[2-(allyloxy)-2-(2,4-dichlorophenyl)ethyl]-1H-imidazole (Imazalil) 35554-44-0
1-[[2-(2,4-dichlorophenyl)-4-propyl-1,3-dioxolan-2-yl]methyl]-1H-1,2,4-triazole (Propiconazole) 60207-90-1
2,2-dibromo-2-cyanoacetamide (DBNPA) 10222-01-2
2,2′,2″-(hexahydro-1,3,5-triazine-1,3,5-triyl)triethanol (HHT) 4719-04-4
2,2′-dithiobis[N-methylbenzamide] (DTBMA) 2527-58-4
2-bromo-2-(bromomethyl)pentanedinitrile (DBDCB) 35691-65-7
2-Butanone, peroxide 1338-23-4
2-butyl-benzo[d] isothiazol-3-one (BBIT) 4299-07-4
2-methyl-2H-isothiazol-3-one (MIT) 2682-20-4
2-octyl-2H-isothiazol-3-one (OIT) 26530-20-1
2-Phenoxyethanol 122-99-6
2-thiazol-4-yl-1H-benzoimidazole (Thiabendazole) 148-79-8
3,3′-methylenebis[5-methyloxazolidine] (Oxazolidin/MBO) 66204-44-2
3-(4-isopropylphenyl)-1,1-dimethylurea/Isoproturon 34123-59-6
3-iodo-2-propynylbutylcarbamate (IPBC) 55406-53-6
4,5-Dichloro-2-octylisothiazol-3(2H)-one (4,5-Dichloro-2-octyl-2H-isothiazol-3-one (DCOIT)) 64359-81-5
5-chloro-2-(4-chlorophenoxy)phenol (DCPP) 3380-30-1
6-(phthalimido)peroxyhexanoic acid (PAP) 128275-31-0
7a-ethyldihydro-1H,3H,5H-oxazolo[3,4-c]oxazole (EDHO) 7747-35-5 Acrolein 107-02-8
Active Chlorine: manufactured by the reaction of hypochlorous acid and sodium hypochlorite produced in situ
Alkyl (C12-16) dimethylbenzyl ammonium chloride (ADBAC/BKC (C12-16)) 68424-85-1
Alkyl (C12-18) dimethylbenzyl ammonium chloride (ADBAC (C12-18)) 68391-01-5
Alkyl (C12-C14) dimethylbenzylammonium chloride (ADBAC (C12-C14)) 85409-22-9
Alkyl (C12-C14) ethylbenzylammonium chloride (ADEBAC (C12-C14)) 85409-23-0
Allyl isothiocyanate 57-06-7
Amines, C10-16-alkyldimethyl, N-oxides 70592-80-2
Amines, N—C12-C14 (even-numbered)-alkyl-trimethylenedi-, reaction products with chloroacetic acid (Ampholyt 20) 139734-65-9
Ammonium bromide 12124-97-9
Ammonium sulphate 7783-20-2
Azoxystrobin 131860-33-8
Benzoic acid 65-85-0
Biphenyl-2-ol 90-43-7
Bis(1-hydroxy-1H-pyridine-2-thionato-O,S)copper (Copper pyrithione) 14915-37-8
Bromine chloride 13863-41-7
Bromoacetic acid 79-08-3
Bromochloro-5,5-dimethylimidazolidine-2,4-dione (BCDMH/Bromochlorodimethylhydantoin) 32718-18-6
Bronopol 52-51-7
Calcium hydroxide (Ca dihydroxide/caustic lime/hydrated lime/slaked lime) 1305-62-0
Calcium hypochlorite 7778-54-3
Calcium magnesium oxide/dolomitic lime 37247-91-9
Calcium magnesium tetrahydroxide (Ca Mg hydroxide/hydrated dolomitic lime) 39445-23-3
Calcium oxide (lime/burnt lime/quicklime) 1305-78-8
Carbendazim 10605-21-7
Cetylpyridinium chloride 123-03-5
Chloramin B 127-52-6
Chlorine 7782-50-5
Chlorine dioxide 10049-04-4
Chlorocresol 59-50-7
Cinnamaldehyde/3-phenyl-propen-2-al (Cinnamic aldehyde) 104-55-2
cis-1-(3-chloroallyl)-3,5,7-triaza-1-azoniaadamantane chloride (cis CTAC) 51229-78-8
Citric acid 77-92-9
Clorophene (Chlorophene) 120-32-1
Copper 7440-50-8
Copper sulphate pentahydrate 7758-99-8
Copper thiocyanate 1111-67-7
Cyanamide 420-04-2
D-gluconic acid, compound with N,N″-bis(4-chlorophenyl)-3,12-diimino-2,4,11,13-tetraazatetradecanediamidine(2:1) (CHDG) 18472-51-0
Decanoic acid 334-48-5
Dichloro-N-[(dimethylamino)sulphonyl]fluoro-N-(ptolyl)methanesulphenamide (Tolylfluanid) 731-27-1
Dicopper oxide 1317-39-1
Didecyldimethylammonium chloride (DDAC (C8-10)) 68424-95-3
Didecyldimethylammonium chloride (DDAC) 7173-51-5
Dimethyloctadecyl[3-(trimethoxysilyl)propyl]ammonium chloride 27668-52-6
Dimethyltetradecyl[3-(trimethoxysilyl)propyl]ammonium chloride 41591-87-1
Disodium peroxodisulphate/Sodium persulphate 7775-27-1
Diuron 330-54-1
Dodecylguanidine monohydrochloride 13590-97-1
Ethanol 64-17-5
Ethylene oxide 75-21-8
Fludioxonil 131341-86-1
Formaldehyde 50-00-0
Formic acid 64-18-6
Glutaral (Glutaraldehyde) 111-30-8
Glycolic acid 79-14-1
Glyoxal 107-22-2
Hexa-2,4-dienoic acid (Sorbic acid) 110-44-1
Hydrochloric acid
Hydrogen peroxide 7722-84-1
Iodine 7553-56-2
L-(+)-lactic acid 79-33-4
Magnesium monoperoxyphthalate hexahydrate (MMPP) 84665-66-7
MBIT 2527-66-4
Mecetronium ethyl sulphate (MES) 3006-10-8
Medetomidine 86347-14-0
Metam-sodium 137-42-8
Methenamine 3-chloroallylochloride (CTAC) 4080-31-3
Methylene dithiocyanate 6317-18-6
Mixture of 5-chloro-2-methyl-2H-isothiazol-3-one (EINECS 247-500-7) and 2-methyl-2H-isothiazol-3-one (EINECS 220-239-6) (Mixture of CMIT/MIT) 55965-84-9
Monohydro chloride of polymer of N,N′″-1,6-hexanediylbis[N′-cyanoguani-dine](EINECS 240-032-4) and hexamethylenediamine (EINECS 204-679-6)/Polyhexamethylene biguanide (monomer: 1,5-bis(trimethylen)-guanylguanidinium monohydrochloride) (PHMB) 27083-27-8
Monolinuron 1746-81-2
N,N′-methylenebismorpholine (MBM) 5625-90-1
N-(3-aminopropyl)-N-dodecylpropane-1,3-diamine (Diamine) 2372-82-9
N-(Dichlorofluoromethylthio)-N′,N′-dimethyl-N-phenylsulfamide (Dichlofluanid) 1085-98-9
N-(trichloromethylthio)phthalimide (Folpet) 133-07-3
Nonanoic acid (Pelargonic acid) 112-05-0
N′-tert-butyl-N-cyclopropyl-6-(methylthio)-1,3,5-triazine-2,4-diamine (Cybutryne) 28159-98-0
Octanoic acid 124-07-2
Ozone 10028-15-6
p-[(diiodomethyl)sulphonyl]toluene 20018-09-1
Pentapotassium bis(peroxymonosulphate) bis(sulphate) 70693-62-8
Peracetic acid 79-21-0
Peroxyoctanoic acid 33734-57-5
Poly(oxy-1,2-ethanediyl),alpha-[2-(dide-cylmethylammonio)ethyl]-omega-hydroxy-, propanoate (salt) (Bardap 26) 94667-33-1
Polymer of N-Methylmethanamine (EINECS 204-697-4 with (chloromethyl) oxirane (EINECS 203-439-8)/Polymeric quaternary ammonium chloride (PQ Polymer) 25988-97-0 Polyvinylpyrrolidone iodine 25655-41-8
Potassium (E,E)-hexa-2,4-dienoate (Potassium Sorbate) 24634-61-5
Potassium 2-biphenylate 13707-65-8
Potassium dimethyldithiocarbamate 128-03-0
Propan-1-ol 71-23-8
Propan-2-ol 67-63-0
Pyridine-2-thiol 1-oxide, sodium salt (Sodium pyrithione) 3811-73-2
Pyrithione zinc (Zinc pyrithione) 13463-41-7
Quaternary ammonium compounds, benzyl-C12-18-alkyldimethyl, salts with 1,2-benzisothiazol-3(2H)-one 1,1-dioxide 68989-01-5
Reaction mass of titanium dioxide and silver chloride
Reaction products of 5,5-dimethylhydantoin, 5-ethyl-5-methylhydantoin with bromine and chlorine (DCDMH)
Reaction products of: glutamic acid and N—(C12-C14-alkyl)propylenediamine (Glucoprotamin) 164907-72-6
Salicylic acid 69-72-7
Silver 7440-22-4
Silver adsorbed on silicon dioxide
Silver copper zeolite 130328-19-7
Silver nitrate 7761-88-8
Silver phosphate glass 308069-39-8
Silver sodium hydrogen zirconium phosphate 265647-11-8
Silver zeolite
Silver zinc zeolite 130328-20-0
Sodium 2-biphenylate 132-27-4
Sodium bromide 7647-15-6
Sodium dichloroisocyanurate dihydrate 51580-86-0
Sodium dimethyldithiocarbamate 128-04-1
Sodium hypochlorite 7681-52-9
Sodium metabisulfite 7681-57-4
Sodium N-(hydroxymethyl)glycinate 70161-44-3
Sodium p-chloro-m-cresolate 15733-22-9
Sulphur dioxide 7446-09-5
Symclosene 87-90-1
tebuconazole 107534-96-3
Terbutryn 886-50-0
Tetrachlorodecaoxide complex (TCDO) 92047-76-2
Tetrahydro-1,3,4,6-tetrakis(hydroxymethyl)imidazo[4,5-d]imidazole-2,5 (1H,3H)-dione (TMAD) 5395-50-6
Tetrahydro-3,5-dimethyl-1,3,5-thiadia-zine-2-thione (Dazomet) 533-74-4
Tetrakis(hydroxymethyl)phosphonium sulphate (2:1) (THPS) 55566-30-8
Thiram 137-26-8
Tosylchloramide sodium (Tosylchloramide sodium-Chloramin T) 127-65-1
Tralopyril 122454-29-9
Triclosan 3380-34-5
Troclosene sodium 2893-78-9
Zineb 12122-67-7;
   also of importance are Pyrithiones, Dimethyldimethylol Hydantoin, Methyichloroisothiazolinone/methylisothiazolinone, Sodium Sulfite, Sodium Bisulfite, Imidazolidinyl Urea, Diazolidinyl Urea, Benzyl Alcohol, 2-Bromo-2-nitropropane-1,3-diol, formaldehyde, lodopropenyl Butylcarbamate, Chloroacetamide, Methanamine, Methyldibromonitrile Glutaronitrile (1,2-Dibromo-2,4-dicyanobutane), Glutaraldehyde, 5-bromo-5-nitro-1,3-dioxane, Phenethyl Alcohol, o-Phenylphenol/s, for example, commonly encountered compounds such as farnesol, parfumes, phenoxyethanol, quaternary compounds, triclocarban, organic acids such as benzoic acid or sorbic acid, biguanides such as poly-(hexamethylene biguanide) hydrochloride phenoxypropanol, benzalkonium chloride, cetrimonium bromide or benzethonium chloride or salicylic acid and the like. Another class of antibacterial agents, which can additionally be used, are the so-called “natural” antibacterial actives, referred to as natural essential oils. Additional active agents are antibacterial metal salts. This class generally includes salts of metals in groups 3b-7b, 8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof.

The invention also pertains to a method for combating harmful organisms or for protecting human beings, animals, materials or processes from the effects of these harmful organisms, wherein the habitat of the harmful organism or the human being, animal or material to be protected is brought into contact with a biocide composition or the biocide composition is employed in said process, wherein the biocide composition comprises at least one polymeric, ionic compound comprising at least 5 imidazolium groups as defined above, provided that the method is not a method for treatment of the human or animal body by surgery or therapy or diagnostic method practised on the human or animal body. “Harmful organism” means any organism, including pathogenic agents, which have an unwanted presence or a detrimental effect on humans, their activities or the products they use or produce, or on animals, plants or the environment. The present method is preferably used for combating harmful organisms selected from bacteria, fungi, algae, viruses and mycoplasma, especially bacteria and fungi.

According to the classification of the EU Biocidal Products Directive, biocides are classified in 23 product types (i.e. application categories), which may roughly be categorised in the following main groups: general biocidal products, disinfectants, preservatives, pest control, anti-fouling products. The present invention includes a product selected from general biocidal products, disinfectants, preservatives, pest control, anti-fouling products, each of which is characterized by containing the present polyimidazolium compound as an antimicrobial agent.

The invention also pertains to a process for achieving an antimicrobial effect, especially an antibacterial and/or antifungal effect, on a hard surface, by contacting said surface with a liquid formulation as described above, which formulation contains the present polyimidazolium compound.

The invention includes the use of the polymeric, ionic compound defined above as a biocide in compositions for various applications, examples of which are
a plant protection composition; preferably a fungicidal composition, or
a personal care composition, or
a home care composition, or
a composition used for industrial or institutional or hospital disinfection,
a material protection composition, or
a pharmaceutical composition.

Further examples are hair-treatment compositions, skin-cleansing compositions, compositions for the care and protection of the skin, nail care compositions and preparations for decorative cosmetics.

Plant Protection

The invention includes a formulation for plant protection, especially for combating harmful fungi. The formulation for plant protection comprises at least one imidazolium polymer as described above, e.g. of formula I, and an agriculturally acceptable carrier and/or adjuvant. The formulation may optionally further contain another active substance for plant protection.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha. In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material. Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system.

Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

Any further active components are, if desired, added in a ratio of from 20:1 to 1:20 to the polymeric, ionic compound(s) comprising imidazolium groups.

In another aspect, the invention relates to a method for combating harmful fungi, which method comprises treating the fungi or materials, plants, parts thereof, the locus where the plants grow or are to grow or plants' propagation material to be protected from fungal attack with an effective amount of at least one polymeric, ionic compound comprising imidazolium groups (imidazolium compound), obtainable as described above.

Further active substances for plant protection, which are optionally contained in the present formulation, are as listed in WO 2012/127009 from page 127, line 21, to page 132 line 7.

Personal Care

The invention accordingly relates also to a personal care preparation comprising at least one imidazolium polymer as described above, e.g. of formula I, and cosmetically tolerable carriers or adjuvants.

The personal care composition typically comprises

A) at least one polymeric, ionic compound comprising imidazolium groups (imidazolium compound) as defined above,
B) optionally at least one cosmetically acceptable active ingredient, and
C) at least one cosmetically acceptable auxiliary.

The composition may, for example, be formulated in the form of
an emulsion, preferably selected from as a W/O, O/W, O/W/O, W/O/W or PIT emulsion and microemulsions,
a suspension,
a gel,
an oil, a cream, milk or lotion,
a powder, a lacquer, a tablet or make-up,
a stick,
a spray or an aerosol,
a foam,
a paste, mousse or an ointment.

The polymers of the invention are accordingly suitable as antimicrobial active substances and preservatives in personal care preparations, including cosmetic products and household products, for example shampoos, bath additives, skin and hair care preparations, liquid and solid soaps, lotions, creams, deodorants and other aqueous or alcoholic solutions. The invention accordingly relates also to a personal care preparation comprising a non-ionic surfactant.

Depending upon the form of the personal care preparation, the amount of imidazolium polymer of the present invention and optionally surfactant will vary and may comprise a majority of the preparation. Typically, the personal care preparation according to the invention contains from about 0.01 to about 30% by weight, for example from about 0.1 to about 20% by weight, for example from about 0.1 to about 15% by weight based on the total weight of the composition, of a mixture of the ionic imidazolium polymer and a surfactant, besides and cosmetically tolerable adjuvants.

The antimicrobial agents of the present invention can be used as ingredients in a wide variety of cosmetic or personal care preparations. Suitable personal care Compositions include skin-care preparations, e.g. skin-washing and cleansing preparations in the form of tablets, liquid soaps, bar soaps, syndets, washing gels, soapless detergents or washing pastes, bath preparations, e.g. liquid (foam baths, milks, oils, shower preparations) or solid bath preparations, e.g. bath cubes and bath salts; skin-care preparations, e.g. skin emulsions, multi-emulsions, powders, sprays or skin oils; cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers; foot-care preparations, e.g. foot baths, foot powders, foot creams or foot balsams, special deodorants and antiperspirants or callus-removing preparations; light-protective preparations, such as sun milks, lotions, creams or oils, sunblocks or tropicals, pre-tanning preparations or after-sun preparations; skin-tanning preparations, e.g. self-tanning creams; depigmenting preparations, e.g. preparations for bleaching the skin or skin-lightening preparations; insectrepellents, e.g. insect-repellent oils, lotions, sprays or sticks; deodorants, such as deodorant sprays, deodorant aerosols, pump-action sprays, deodorant gels, sticks or rollons, also water-free deodorant aerosols or sticks; antiperspirants, e.g. antiperspirant sticks, creams or roll-ons, also water-free antiperspirant aerosols and water-free antiperspirant sticks; preparations for cleansing and caring for blemished skin, e.g. synthetic detergents (solid or liquid), peeling or scrub preparations or peeling masks; hair-removal preparations in chemical form (depilation), e.g. hair-removing powders, liquid hair-removing preparations, cream- or paste-form hair-removing preparations, hair-removing preparations in gel form or aerosol foams; shaving preparations, e.g. shaving soap, foaming shaving creams, non-foaming shaving creams, foams and gels, pre-shave preparations for dry shaving, aftershaves or aftershave lotions; fragrance preparations, e.g. fragrances (eau de Cologne, eau de toilette, eau de parfum, parfum de toilette, perfume), perfume oils or perfume creams; cosmetic hair-treatment preparations, e.g. hairwashing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hairsetting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile, antidandruff preparations in the form of shampoos, conditioners, hair tonics, styling creams or gels or treatments packs; oral care preparations such as pastes, gels, powders, mouth washes and sprays.

Especially preferred are the preparations like skin-care preparations, bath preparations, cosmetic personal care preparations, foot-care preparations; light-protective preparations, skin-tanning preparations, depigmenting preparations, insect-repellents, deodorants, antiperspirants, preparations for cleansing and caring for blemished skin, hair-removal preparations in chemical form (depilation), shaving preparations, fragrance preparations or cosmetic hair-treatment preparations.

In order to achieve the mildness required of the antimicrobial composition of the present invention, optional ingredients to enhance the mildness to the skin can be added. These ingredients are well known in the filed and include synthetic and naturally occurring polymers, co-surfactants and moisturizers.

For example, lipophilic skin conditioning agents may be present, for example, hydrocarbon oils and waxes, silicones, fatty acid derivatives, cholesterol, cholesterol derivatives, di- and tri-glycerides, vegetable oils, vegetable oil derivatives, liquid nondigestible oils such as those described in U.S. Pat. Nos. 3,600,186; 4,005,195 and 4,005,196, all of which are herein incorporated by reference, or blends of liquid digestible or nondigestible oils with solid polyol polyesters such as those described in U.S. Pat. Nos. 4,797,300; 5,306,514; 5,306,516 and 5,306,515, all of which are herein incorporated by reference, and acetoglyceride esters, alkyl esters, alkenyl esters, lanolin and its derivatives, milk tri-glycerides, wax esters, beeswax derivatives, sterols, phos-pholipids and mixtures thereof.

Nonlimiting examples of silicone useful herein are described in U.S. Pat. No. 5,011,681, to Ciotti et al., issued Apr. 30, 1991, which is incorporated by reference.

Nonlimiting examples of naturally occurring oils include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils and vegetable oil derivatives; coconut oil and derivatized coconut oil, cottonseed oil and derivatized cottonseed oil, jojoba oil, cocoa butter, and the like.

Acetoglyceride esters may be used and an example is acetylated monoglycerides.

When a lipophilic skin moisturizing agent is employed as the mildness enhancer in the antimicrobial compositions herein, a stabilizer may also be included. The stabilizer is used to form a crystalline stabilizing network in the liquid composition that prevents the lipophilic skin moisturizer agent droplets from coalescing and phase splitting in the product. The network exhibits time dependent recovery of viscosity after shearing (e.g., thixotropy).

The stabilizers used herein are not surfactants. The stabilizers provide improved shelf and stress stability. Typical hydroxyl-containing stabilizers include 12-hydroxystearic acid, 9,10-dihydroxystearic acid, tri-9,10-dihydroxystearin and tri-12-hydroxystearin (hydrogenated castor oil is mostly tri-12-hydroxystearin).

Alternatively, the stabilizer employed in the antimicrobial compositions herein can comprise for example a polymeric thickener, a C₁₀-C₂₂ ethylene glycol fatty acid ester, dispersed amorphous silica, dispersed smectite clay such as bentonite and hectorite, a fatty acid or fatty alcohol.

The antimicrobial compositions of the present invention can comprise a wide range of optional ingredients. The CTFA International Cosmetic Ingredient Dictionary, Sixth Edition, 1995, which is incorporated by reference herein in its entirety, describes a wide variety of materials commonly used in the cosmetic and personal care industry suitable for use in the compositions of the present invention. Nonlimiting examples of functional classes of ingredients are described at page 537 of this reference.

Examples of these functional classes include: abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include solubilizing agents, sequestrants, and keratolytics, and the like.

The personal care preparation according to the invention may exist in a wide variety of presentation forms, for example a water-in-oil or oil-in-water emulsion, an alcoholic or alcohol-containing formulation, a vesicular dispersion of an ionic or non-ionic amphiphilic lipid, a gel, a solid stick, cream, milk or lotion, a powder, a lacquer, a tablet or make-up, a stick, a spray or an aerosol, a foam, or a paste and all kinds of microemulsions.

As a water-in-oil (W/O) or oil-in-water emulsion (O/W), the cosmetically tolerable adjuvant typically contains from 5 to 50% of an oil phase, from 5 to 20% of an emulsifier and from 30 to 90% water. The oil phase may comprise any oil suitable for cosmetic formulations, for example one or more hydrocarbon oils, a wax, a natural oil, a silicone oil, a fatty acid ester or a small chain or fatty alcohol including mono- and poly-ols, e.g., ethanol, isopropanol, propylene glycol, hexylene glycol, glycerol and sorbitol.

The aqueous phase contains for example ingredients such as alcohols, diols or polyols or their ethers as well as one or more thickeners for example of the groups of silicium dioxide, aluminium silicates, polysaccharides or derivatives thereof for example hyaluronic acid, xanthan gum, hydroxypropylmethylcellulose, polyacrylates.

The compositions of the invention may also include various thickeners, such as crosslinked acrylates, nonionic polyacrylamides, xanthan gum, guar gum, gellan gum, and the like; polyalkyl siloxanes, polyaryl siloxanes, and aminosilicones.

The specific examples of the suitable thickening silicon compounds include polydimethylsiloxane, phenylsilicone, polydiethylsiloxane, and polymethylphenylsiloxane. Some of the suitable silicon compounds are described in European Patent Application EP 95,238 and U.S. Pat. No. 4,185,017, which patent is incorporated herein by reference. The compositions of the invention may also include silicone polymer materials, which provide both style retention and conditioning benefits to the hair. Such materials are described in U.S. Pat. No. 4,902,499, which is incorporated herein by reference.

Further cosmetically tolerable adjuvants include emulsifiers, perfume oils, rheology modifiers (thickeners), hair polymers, hair and skin conditioners, water-soluble or dispersible silicone-comprising polymers, bleachers, gelling agents, care agents, colorants, tinting agents, tanning agents, dyes, pigments, antidandruff agents, sunscreen agents, deodorizing active substances, vitamins, plant extracts, bodying agents, humectants, refatting agents, collagen, protein hydrolysates, lipids, antioxidants, antifoaming agents, antistatic agents, emollients, softeners, etc.

Further suitable cosmetically active substances for use within the composition of the invention are, for example, skin and hair pigmentation agents, tanning agents, bleaches, keratin-hardening substances, antimicrobial active substances, photofilter active substances, repellent active substances, hyperemic substances, keratolytic and keratoplastic substances, antidandruff active substances, antiphlogistics, keratinizing substances, active substances which act as antioxidants and/or as free-radical scavengers, skin moisturizing or humectant substances, refatting active substances, deodorizing active substances, sebostatic active substances, plant extracts, antierythimatous or antiallergic active substances and mixtures thereof; artificially skin-tanning active substances which are suitable for tanning the skin without natural or artificial irradiation with UV rays are, for example, dihydroxyacetone, alloxan and walnut shell extract; suitable keratin-hardening substances are generally active substances as are also used in antiperspirants, such as, for example, potassium aluminum sulfate, aluminum hydroxychloride, aluminum lactate, etc.; further antimicrobial active substances are as defined further above, these include, for example, customary preservatives known to the person skilled in the art, such as phydroxybenzoates, imidazolidinylurea, formaldehyde, sorbic acid, benzoic acid, salicylic acid, etc.; deodorizing substances are, for example, zinc ricinoleate, triclosan, undecylenic acid alkylolamides, triethyl citrate, chiorhexidine etc.; suitable photofilter active substances are substances which absorb UV rays in the UV-B and/or UV-A region; suitable UV filters include hydroxyphenyl triazines, p-aminobenzoic esters, cinnamic esters, benzophenones, camphor derivatives, and pigments which stop UV rays, such as titanium dioxide, talc and zinc oxide; suitable repellent active substances are compounds which are able to keep or drive certain animals, in particular insects, away from people, these include, for example, 2-ethyl-1,3-hexanediol, N,N-diethyl-m-toluamide, etc.; suitable hyperemic substances which stimulate blood flow in the skin are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc.; suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenol, etc.

The compositions according to the invention can comprise at least one further polymer. These include, very generally, anionic, cationic, amphoteric and neutral polymers. Suitable anionic polymers for the personal care compositions according to the invention are generally all anionic polymers known for this application. The composition according to the invention preferably comprises at least one soluble or dispersed anionic polymer. The anionic polymers that may be employed include, but are not limited to, polymers comprising groups derived from carboxylic acids, sulfonic acids or phosphoric acids. Preferably, the anionic polymers have a number-average molecular mass in a range from 500 to 5 000 000.

Suitable additional cationic polymers are polymers different from the imidazolium compounds according to the invention; they may be chosen in principle from all cationic polymers known to a person skilled in the art as suitable for cosmetic compositions. Cationic polymers for compositions for improving the cosmetic properties of the hair are for example those described in patent applications EP-A-0 337 354 FR-A-2 270 846, FR2 383 660, FR-2 598 611, FR-2 470 596 and FR-2 519 863. For the purposes of the present invention, the term “cationic polymer” denotes any polymer comprising at least one cationic group or at least one cationogenic group that may be ionized into a cationic group. The at least one cationic polymer may be chosen from those containing units comprising primary, secondary, tertiary, and/or quaternary amine groups that either may form part of the main polymer chain or may be borne by a side substituent directly attached thereto.

The personal care composition according to the invention can also be a composition that contains the present imidazolium compound as preservative.

In addition to the abovementioned constituents, the personal care compositions according to the invention may also comprise at least one surface-active substance, including dispersing agents and wetting agents, besides surfactants already described above.

### Home Care

The invention includes a home care preparation comprising at least one imidazolium polymer as described above, e.g. of formula I, and suitable carriers and/or adjuvants.

The composition optionally contains at least one further microbicidal compound different from the present polyimidazolium compound and/or optionally at least one further active ingredient and/or auxiliary. The home care composition according to the invention can be a composition that is effective against various microorganisms. According to this variant, the imidazolium compound itself may act as active ingredient. Accordingly, in such a composition the use of a further active ingredients and/or auxiliaries is only optional. The home care composition according to the invention can also be a composition that contains the present imidazolium compound as preservative.

A typical home care composition according to the invention contains the present imidazolium compound, optionally at least one further microbicidal compound, and at least one further component selected from non-ionic surfactants, anionic surfactants, amphoteric surfactants, water, alcohols and a combination thereof. The home care compositions can include additional components such as enzymes, bleaches, whiteners, color care agents, fabric softeners, suds suppressors, dispersants, dye transfer inhibitors, chelating agents, aerosol propellants, gelling agents, thickening agents and a combination thereof.

The home care composition according to the invention can be formulated in a variety of ways and may include a hydrophilic phase, a hydrophobic phase and optionally at least one emulsifying agent. The home care composition may be in the form of a liquids, semi-solid, paste, gel, bar, tablet, spray, foam, powder or granules. For the purpose of the invention the term “home care composition” means a composition for use in the general environment of human beings and is further described in the following. Home care compositions are generally nontoxic when applied in the vicinity of human beings, for example to fabrics and other items used by humans, when applied to surfaces used by, or in the vicinity of, humans, or when applied to spaces occupied by humans.

A further aspect of the invention is a method of using a home care composition, as defined above and in the following, by applying the composition to an article, surface or space. Exemplary articles, surfaces and spaces include clothes, furniture fabrics, rugs and carpets, draperies, dishes and cooking utensils, grills, ovens, and other items used by humans. The term “surface” includes hard surfaces in the human environment, such as floors, glass surfaces (such as glass windows, doors and countertops), other counter surfaces, bath, toilet bowl, sink and other bathroom surfaces. The term “space” includes the interior portion of buildings occupied by humans, including the air contained therein.

Advantageously, a home care composition comprising at least one imidazolium compound of the invention possesses effective antimicrobial preservative properties. Further, a home care composition comprising the imidazolium compound also confers an antimicrobial effect on articles, surfaces or spaces to which it is applied. Home care compositions according to the invention include:
surface cleaning compositions (for example, glass, floor, counter, bath, toilet bowl, sink, appliance and furniture cleaning compositions);
deodorants (for example, solid, liquid and spray deodorants air and/or surface deodorants);
disinfectants (for example, spray and solid air disinfectants (including gel); and spray, solid, liquid and paste surface disinfectants);
waxes and other surface protecting and/or polishing compositions;
laundry compositions (for example detergents, fabric softeners and whiteners);
rug shampoos.

Typical products comprising the present imidazolium polymer in the Home Care area include for example hard surface cleaners, floor cleaners, hard surface or floor disinfectants, all purpose cleaner, dish washing agents, bathroom cleaners or hand disinfectants.

The compositions and products of the invention may advantageously be used also for industrial or institutional or hospital disinfection, cleaning and sanitation including Health Care, Building Care, Food service & kitchen hygiene, Food and beverage processing or Laundry Care including hard surface cleaners and disinfectants, floor cleaners and disinfectants, hand disinfectants and soaps, CIP-cleaning products or fabric softeners.

Preferably, the home care composition comprises the present imidazolium polymer and, if present, further adjuvants or active components in a fraction of from about 0.001 to 50% by weight, particularly preferably 0.01 to 30% by weight, in particular 0.1 to 20% by weight, based on the total weight of the composition. Such formulations may be ready-for-use compositions or concentrates, and typically comprise further additional components as described above. Concentrated formulations of the present imidazolium polymer contain it preferably in amounts ranging from 10 to about 70 percent, by total weight of the concentrate.

Pharmaceutical Composition

A further aspect of the invention is a pharmaceutical composition comprising

A) at least one imidazolium compound as defined above,
B) optionally at least one further microbicidal compound different from the compounds of component (A),
C) optionally at least one pharmaceutically acceptable active ingredient, and
D) optionally at least one pharmaceutically acceptable excipient.

The pharmaceutical composition typically comprises the present imidazolium compound as defined above (A), at least one pharmaceutically acceptable carrier or excipient (D), and, optionally, at least one pharmaceutically active ingredient (C) and/or further microbicidal compound different from the present imidazolium compounds (B).

Preferably, the pharmaceutical composition comprises the components A) and, if present, B) in a fraction of from about 0.001 to 50% by weight, particularly preferably 0.01 to 30% by weight, in particular 0.1 to 20% by weight, based on the total weight of the composition.

The pharmaceutical composition of the invention is suitable for administering in principle any type of active pharmaceutical ingredient C). These include benzodiazepines, antihypertensives, vitamins, cytostatics, in particular taxol, anesthetics, neuroleptics, antidepressants, antibiotics, antimycotics, fungicides, chemotherapeutics, urologies, thrombocyte aggregation inhibitors, sulfonamides, spasmolytics, hormones, immunoglobulins, sera, thyroid therapeutic agents, psychopharmacological agents, antiparkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, antiinflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

Examples of suitable active ingredients C) are: acarbose, non-steroidal antirheumatics, cardiac glycosides, acetylsalicylic acid, virustatics, aclarubicin, acyclovir, cisplatin, actinomycin, alpha- and beta-sympathomimetics, allopurinol, alosetron, alprostadil, prostaglandins, amantadine, ambroxol, amlodipine, methotrexate, 5-aminosalicylic acid, amitriptyline, amlodipine, amoxicillin, anastrozole, atenolol, atorvastatin, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, celetoxib, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytarabine, dapiprazole, desogestrel, desonide, dihydralazine, diltiazem, ergot alkaloids, dimenhydrinate, dimethyl sulfoxide, dimethicone, dipyridamole, domperidone and domperidone derivatives, donepzil, dopamine, doxazosin, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenytoin, riluzoles, risedronate, sildenafil, topiramate, macrolide antibiotics, esomeprazole, estrogen and estrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitor, fluconazole, fludarabine, flunarizine, fluorouracil, fluoxetine, flurbiprofen, ibuprofen, flutamide, fluvastatin, follitropin, formoterol, fosfomicin, furosemide, fusidic acid, galantamine, gallopamil, ganciclovir, gemfibrozil, gentamicin, ginkgo, St John's wort, glibenclamide, urea derivatives as oral antidiabetics, glucagon, glucosamine and glucosamine derivatives, glutathione, glycerol and glycerol derivatives, hypothalamus hormones, goserelin, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixen, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline, and adrenaline derivatives, norfloxacin, novaminsulfone, noscapine, nystatin, ofloxacin, olanzapine, olsalazine, omeprazole, omoconazole, ondansetron, orlistat, oseltamivir, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexol, pravastatin, prazosin, procaine, promazine, propiverine, propranolol, propyphenazone, prostaglandins, protionamide, proxyphylline, quetiapine, quinapril, quinaprilate, ramipril, ranitidine, reproterol, reserpine, ribavirin, rifampicin, risperidone, ritonavir, ropinirol, rosiglitazone, roxatidine, roxithromycin, ruscogenin, rutoside and rutoside derivatives, sabadilla, salbutamol, salmeterol, scopolamine, selegiline, sertaconazole, sertindole, sertraline, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, taliolol, tamoxifen, taurolidine, tazarotene, tegaserod, temazepam, teniposide, tenoxicam, terazosin, terbinafine, terbutaline, terfenadine, terlipressin, tertatolol, tetracyclines, tetryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antiestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutin, tulobuterol, tyramine, tyrothricin, urapidil, ursodeoxycholic acid, chenodeoxycholic acid, valaciclovir, valdecoxib, valproic acid, vancomycin, vecuronium chloride, venlafaxine, verapamil, vidarabine, vigabatrine, viloxazine, vinblastine, vincamine, vincristine, vindesine, vinorelbine, vinpocetine, viquidit, warfarin, xantinol nicotinate, xipamide, zafirlukast, zalcitabine, zanamivir, zidovudine, zolmitriptan, Zolpidem, zopiclone, zotepine and the like.

The active ingredients can, if desired, also be used in the form of their pharmaceutically acceptable salts or derivatives, and in the case of chiral active ingredients it is possible to employ both optically active isomers and racemates or mixtures of diastereoisomers. The compositions of the invention can, if desired, also comprise two or more active pharmaceutical ingredients.

The formulation base of pharmaceutical compositions of the invention preferably comprises pharmaceutically acceptable excipients D). Pharmaceutically acceptable excipients are those known to be usable in the area of pharmacy, food technology and adjacent sectors, in particular the excipients listed in relevant pharmacopeias (e.g. DAB, Ph. Eur., BP, USP, JP) and others, whose properties do not stand in the way of physiological use. Suitable excipients D) may be: lubricants, wetting agents, emulsifying and suspending agents, antioxidants, anti-irritants, chelating agents, emulsion stabilizers, film formers, gel formers, odor-masking agents, resins, hydrocolloids, solvents, solubilizers, neutralizers, permeation promoters, pigments, colorants, stabilizers, disintegrants, dessicants, opacifiers, thickeners, waxes, plasticizers, flavors, sweeteners, excipients to reduce permeation etc. An arrangement concerning this is based on specialist knowledge as described for example in Fiedler, H. P. Lexikon der Hilfsstoffe fur Pharmazie, Kosmetik und angrenzende Gebiete, 4th edition, Aulendorf: ECV-Editio-Cantor-Verlag, 1996.

Antimicrobial formulations of the invention are also applied in clean-in-place (CIP) applications. CIP applications are used in practice in the food and beverage industry, like in breweries, in the dairy industry, in the soft-drink and juice manufacturing industry, but also in the cosmetic and pharmaceutical industry.

As noted, the invention includes a product selected from the group consisting of home care formulation, a disinfectant of hard and/or soft surfaces, sanitary detergent of hard and/or soft surfaces, and product for clean in place (CIP), wherein said article comprises, preferably comprises at least 70 wt.-% of, still more preferably comprises at least 90 wt.-% of, yet more preferably consists of, the formulation as described herein. Accordingly the present invention is especially directed to a product selected from the group consisting of disinfectant, all purpose cleaner, dishwashing liquid, descaling agent, bath room cleaner, toilet bowl cleaner, floor cleaner, glass cleaner, kitchen cleaner, sanitary cleaner, furniture cleaner and product for clean in place (CIP), wherein said product comprises, preferably comprises at least 70 wt.-% of, still more preferably comprises at least 90 wt.-% of, yet more preferably consists of, the formulation as described herein.

Like the formulation also the products containing the formulation of the present invention can be used as concentrate that has to be diluted with water prior to use or are ready-to-use products, which are used as such, e.g. without dilution.

The terms used in the previous paragraphs are understood according to the knowledge of the skilled artisan in the respective technical field. For instance a disinfectant is a substance that is applied to non-living (i.e. inanimate) objects to destroy microorganisms that are living on said objects. On the other hand the clean in place is a method of cleaning and/or disinfecting the interior surfaces of pipes, vessels, process equipment, filters and associated fittings without disassembly. The clean in place product according to this invention is especially used for removing soils and disinfection in facilities for processing typically liquid product streams such as beverages, milk, juices, etc. The formulation of the invention can be used for disinfection of the interior surfaces in CIP after a separate cleaning step. The cleaning step is performed with a cleaning formulation that is not biocidal or disinfecting. It may however also be the case that the formulation of the invention is used for CIP-cleaning and CIP disinfection in one single step.

Depending on the end application the formulation may contain further ingredients making it ready for the desired end-use. Such ingredients are known to the skilled artisan and do not contribute to the present invention. Depending upon the form of the formulation used at the end it comprises, in addition to the components mentioned above further constituents, for example surfactants (surface active agents), pH regulators, buffering agents, hydrotropes, polymers, metal sequestering or metal chelating agents, colourings, perfume oils, thickening or solidifying agents (consistency regulators), emulsifiers, emollients, UV-absorbers, and antioxidants.

Within the context of the invention, cleaning compositions or cleaners are:
compositions for cleaning and/or disinfecting hard surfaces, such as all-purpose cleaners, floor cleaners, dishwashing detergents for manual or automatic dishwashing, hand washes, glass cleaners, kitchen cleaners, bath and sanitary cleaners, WC cleaners, disinfectant cleaners,
cleaning and care compositions for textiles and laundry such as detergents, fabric softeners, stain removers.

Formulation types of the cleaners are selected from the group comprising: cleaner and disinfectant concentrate, liquid cleaners or disinfectants, pulverulent cleaners or disinfectants, sprays for cleaning or disinfection, emulsions and gels.

The surfaces to be treated are selected from the group comprising: glass, plastics, metal, steel, wood, stone materials, ceramic, cement, coatings, composite materials, textiles (natural fibers such as e.g. cotton, wool, silk and synthetic fibers such as polyester, polyolefins (PE, PP etc.), polyamide, polyurethane, PVC etc.), foam materials and upholstery materials and carpets.

In one variant of the present invention, it is a cleaning composition for hard surfaces. Within the context of the invention, hard surfaces are e.g. tiles, ceramic, glass, glass fibers, metals, steel, aluminum, plastic, wood, stone materials, coatings, composite materials, cement and the like, but no textiles.

During the cleaning of hard surfaces, it is often necessary to disinfect a surface. In this application, disinfection is understood as meaning the killing of microorganisms or the reduction in the growth of microorganisms.

The present polyimidazolium compounds may also be used as preservatives for material protection. These preservatives are preferably used in cosmetic and/or pharmaceutical formulations.

Furthermore, the polymers of the invention can also be used for control of microorganisms in technical applications as in-can preservative for technical products and/or preservative for aqueous/water containing systems and processes.

In contrast to the disinfection agents, which destroys the microorganism very fast, a preservative has to have an effect over a longer period of time. Preservative have a so called microbiostatic effect.

The concentration of the preservative according to the present invention in a formulation can vary. Generally, the preservatives of the present invention can be incorporated in a commercial formulation at a concentration of between about 0.01 and about 5 weight percentage (wt-%), based on total formulation. More specifically, for cosmetic or pharmaceutical formulations, the amount of the present mixture need not exceed 1.0 wt-%; however, for industrial or household cleaners, up to 5 wt-% can be employed when desired.

An embodiment of the present invention is a personal care formulation comprising 0.01 to 5 wt-% of at least one polyimidazolium compound, such as a compound of formula (I), and 95 to 99.99 wt-% of at least one cosmetically tolerable adjuvant.

Another embodiment of the present invention is a pharmaceutical formulation comprising 0.01 to 5 wt-% of at least one polyimidazolium compound, e.g. of formula (I), and 95 to 99.99 wt-% of at least one pharmaceutically tolerable adjuvant.

Another embodiment of the present invention is a household formulation comprising at least one polyimidazolium compound, e.g. of formula (I), and adjuvants used in household formulations.

A soap has, for example, the following composition:

| | | |
|---|---|---|
| 0.01 to | 5 wt-% | of at least one compound of formula (I), |
| 0.3 to | 1 wt-% | of titanium dioxide, |
| 1 to | 10 wt-% | of stearic acid, |
| 0 to | 10 wt-% | of at least one auxiliary, and |

| | |
|---|---|
| ad 100 % | of a soap base, e.g. the sodium salts of tallow fatty |
| | acid and coconut fatty acid or glycerol. |

A shampoo has, for example, the following composition:

| | | |
|---|---|---|
| 0.01 to 5 | wt-% | of at least one compound of formula (I), |
| 12.0 | wt-% | of sodium laureth-2-sulfate, |
| 4.0 | wt-% | of cocamidopropyl betaine, |
| 3.0 | wt-% | of NaCl, |
| 0 to 10 | wt-% | of at least one auxiliary, and |
| ad 100 | wt-% | of water. |

A deodorant has, for example, the following composition:

| | | |
|---|---|---|
| 0.01 to 5 | wt-% | of at least one compound of formula (I), |
| 60 | wt-% | of ethanol, |
| 0.3 | wt-% | of perfume oil, |
| 0 to 10 | wt-% | of at least one auxiliary, and |
| ad 100 | wt-% | of water. |

All wt-%'s are based on the total weight of the compositions.

The invention relates also to an oral composition, comprising from 0.01 to 15 wt-%, based on the total weight of the composition, of at least one compound of formula (I), and orally tolerable adjuvants.

Example of an oral composition:

| | | |
|---|---|---|
| 10 | wt-% | of sorbitol |
| 10 | wt-% | of glycerol |
| 15 | wt-% | of ethanol |
| 15 | wt-% | of propylene glycol |
| 0.5 | wt-% | of sodium lauryl sulfate |
| 0.25 | wt-% | of sodium methylcocyl taurate |
| 0.25 | wt-% | of polyoxypropylene/polyoxyethylene block copolymer |
| 0.10 | wt-% | of peppermint flavouring |
| 0.1 to 0.5 | wt-% | of at least one compound of formula (I), and |
| 48.6 | wt-% | of water. |

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name Olafluor.

The present compounds, e.g. those of formula (I), can be used also in household and all-purpose cleaners for cleaning hard surfaces. A cleaning preparation has, for example, the following composition:

| | | |
|---|---|---|
| 0.01 to 5 | wt-% | of at least one compound of formula (I), and |
| 3.0 | wt-% | octyl alcohol 4EO |
| 1.3 | wt-% | fatty alcohol C₈-C₁₀polyglucoside |
| 3.0 | wt-% | isopropanol |
| 0 to 5 | wt-% | of auxiliaries, |
| ad 100 | wt-% | water. |

Also possible, in addition to the preservation of cosmetics, personal care and household products is the use of the inventive biocide in controlling microorganisms in technical products as well as in industrial and water containing systems and processes such as cooling towers, heat exchangers, boiler systems, other industrial process water, ballast water, wastewater treatment systems, reverse osmosis water processing but also swimming pools and spa facilities, pulp and paper manufacture, metal working fluids, leather manufacture, paints and coatings, aqueous emulsions, latexes, adhesives, inks, synthetic and protein-based glues, pigment dispersions, mineral slurries, caulks and adhesives, tape joint compounds, disinfectants, cleaners, textile fluids, or a system used therewith.

Moreover, the biocides of the invention are useful for controlling microorganisms in aqueous or water-containing systems, such as those present in oil and natural gas applications. Examples of such systems include, but are not limited to, injection and produced water, source water for waterflooding and hydraulic fracturing such as pond water and holding tank water, functional fluids such as drilling muds, workover fluids, hydrotest fluids, packer fluids, oil and gas pipelines or fuel.

In addition, the biocides may be employed in other areas. The biocides of the invention are suitable for use over a wide temperature range. In a preferred embodiment, the biocides are used in aqueous or water-containing systems at a temperature of 20° C. or greater (e.g. within the temperature range 20-90° C.). In further embodiments, the temperature of the aqueous or water containing system is 60° C. or greater, or is 80° C. or greater. The biocides are also further effective when a reducing agent such as a source of sulfide ion is present in the aqueous or water-containing system.

The formulation of the invention is typically used, for example, as

(a) a home care formulation, such as a disinfectant, all purpose cleaner, dishwashing liquid, descaling agent, a bath room cleaner, a toilet bowl cleaner,
and/or
(b) a disinfectant and/or sanitary detergent of hard and/or soft surfaces, such as a floor cleaner, a glass cleaner, a kitchen cleaner, a bath room cleaner, a sanitary cleaner, a toilet bowl cleaner, a furniture cleaner,
and/or
(c) a product for clean in place,
or for the manufacture of said home care formulation (a), disinfectant and/or sanitary detergent (b), and/or a product for clean in place (c).

In a further aspect the present invention is directed to a product comprising the formulation according to this invention, wherein said product is selected from the group consisting of home care formulation, disinfectant of hard and/or soft surfaces, sanitary detergent of hard and/or soft surfaces, and product for clean in place application.

The invention thus further provides a product selected from the group consisting of home care formulation, disinfectant of hard and/or soft surfaces, sanitary detergent of hard and/or soft surfaces, product for clean in place, all purpose cleaner, dishwashing liquid, descaling agent, bath room cleaner, toilet bowl cleaner, floor cleaner, glass cleaner, kitchen cleaner, sanitary cleaner, furniture cleaner, wherein said product comprises a formulation containing the polymeric, ionic compound comprising at least 5 imidazolium groups and containing alkyl moieties as end groups, as described above.

Formulations for certain application fields such as home care, personal care, sanitary disinfection, hand disinfection, and especially for hospital disinfection, are advantageously applied as liquid formulations by means of a suitable dispenser, which allows applying the required dosage of the formulation. Formulations applied in this manner typically comprise the imidazolium polymer as described above (e.g. of the formula I), a liquid carrier (typically water and/or one or more alcohols, where the alcohol may also possess antimicrobial properties), and optionally further components, which are typically selected from surfactants and further antimicrobial agents.

The term “polymer”, “polymeric ionic compound”, “polyimidazolium compound”, “imidazolium polymer”, “antimicrobial polymer”, and “polymer compound”, as used within this specification including the following examples, denotes the polymeric salts of the invention, including those of the formula I.

The following examples illustrate the invention. Unless indicated otherwise, reactions take place at standard conditions, i.e. atmospheric pressure and room temperature (r.t.), which depicts a temperature from the range 22-25° C.; over night means a period of 12 to 15 hours; percentages are given by weight (b.w.), if not indicated otherwise.

### Abbreviations

Mw molecular weight (weight average, usually as detected by GPC)
Mn molecular weight (number average, usually as detected by GPC)
M concentration in moles per liter
GPC gel permeation chromatography
PDI polydispersity (ratio Mw/Mn)

### Preparation of Polymer A:

2 Mol of acetic acid and 50 g of water are placed in flask. A mixture of 1.0 mol formaldehyde (49% aq. solution) and 1.0 mol glyoxal (40% aq. Solution) is added via a dropping funnel to the solution. In parallel, a mixture of 0.92 mol of 1,6-diaminohexane and 0.17 mol of 1-dodecylamine in 50 g of water is added to the solution via a separated dropping funnel. During addition of the monomers, the reaction mixture is held at room temperature by ice bath cooling. After completion of the addition, the reaction mixture is heated to 100° C. for 1 hour. The crude product (containing 50.5% b.w. of Polymer A) is used as received.

### Preparation of Polymer B:

2 Mol of acetic acid and 50 g of water are placed in flask. A mixture of 1.0 mol formaldehyde (49% aq. Solution) and 1.0 mol glyoxal (40% aq. solution) is added via a dropping funnel to the solution. In parallel, a mixture of 0.92 mol of 1,6-diaminohexane and 0.17 mol of 1-dodecylamine in 50 g of water is added to the solution via a separated dropping funnel. During addition of the monomers, the reaction mixture is held at room temperature by ice bath cooling. After completion of the addition the reaction mixture is heated to 100° C. for 1 hour. After cooling to room temperature, water is distilled off the reaction mixture (100 g). The distilled product (containing 70.3% b.w. of Polymer B) is used as received.

### Preparation of Polymer C:

2 Mol of acetic acid and 50 g of water are placed in flask. A mixture of 1.0 mol formaldehyde (49% aq. solution) and 1.0 Mol glyoxal (40% aq. Solution) and is added via a dropping funnel to the solution. In parallel, a mixture of 1.0 mol of 1,6-diaminohexane and 0.002 mol of 1-dodecylamine in 50 g of water is added to the solution via a separated dropping funnel. During addition of the monomers, the reaction mixture is held at room temperature by ice bath cooling. After completion of the addition the reaction mixture is heated to 100° C. for 1 hour. The crude product (containing 46.6% b.w. of Polymer C) is used as received.

### Preparation of Polymer D:

14 Mol of acetic acid and 350 g of water are placed in flask. A mixture of 7.0 mol formaldehyde (as 49% aq. Solution) and 7.0 mol glyoxal (40% aq. solution) is added via a dropping funnel to the solution. In parallel, a mixture of 6.42 mol of 1,6-diaminohexane and 1.16 mol of 1-dodecylamine in 350 g of water is added to the solution via a separated dropping funnel. During addition of the monomers, the reaction mixture is held at room temperature by ice bath cooling. After completion of the addition the reaction mixture is heated to 100° C. for 1 hour. After cooling to room temperature, water is distilled off the reaction mixture (700 g). The concentrated product is diluted with water to contain 50% b.w. of Polymer D; this solution is used for the tests described below.

### Preparation According to WO 2012/127009(Comparison):

Polymers according to examples 1 (polymer C1) and 10 (polymer C10) of WO 2012/127009 are prepared for comparison purposes, using acetic acid, formaldehyde, glyoxal and 1,4-diaminobutane (C1) or 1,6-diaminohexane (C10), in the absence of any monoaminoalkane.

### GPC-Analysis:

The molecular weight of the polymeric ionic compounds is determined by Size-exclusion chromatography (SEC) using poly(2-vinylpyridine) as a standard, and water comprising 0.1% (w/w) trifluoroacetate and 0.1 M aq. NaCl as effluent. The temperature of the column is 35° C., the injected volume 100 μL (microliter), the concentration 1.5 mg/mL and the flow rate 0.8 mL/min.

The weight average molecular weight (Mw), the number average molecular weight (Mn) and the polydispersity PDI (Mw/Mn) of the polymeric ionic compounds obtained are listed in the following table 1.

TABLE 1
Results of GPC analysis

| Polymer | Mw | Mn | PDI |
|---|---|---|---|
| A | 9000 | 1500 | 6.2 |
| B | 12100 | 1600 | 7.8 |
| C | 95200 | 5340 | 17.8 |
| D | 8540 | 1420 | 6.0 |
| C1 | | | |
| C10 | 5300 | 1460 | 3.6 |

For biological activity testing, the following microorganisms are used:

Staphylococcus aureus (S. aureus ATCC 6538);
Escherichia Coli (E. coli DSM 682);
Pseudomonas aeruginosa (P. aeruginosa ATCC 15442);
Candida albicans (C. albicans DSM 1386 or ATCC 10231, as indicated);
Aspergillus brasiliensis (A. brasiliensis DSM 1988);
Klebsiella pneumoniae (DSM 789).

### Evaluation of Minimal Inhibitory Concentration (MIC):

The polymer of the invention (polyimidazolium salt, PIMS) is dissolved in water and added to Müller-Hinton-Broth (for bacteria) or Malt extract broth (for fungi) at a dilution of 1:50. After inoculation with the microorganism, samples are incubated at 37° C. (in case of bacteria) or 30° C. (in case of fungi) for 48 h and evaluated with regard to turbidity of the broth medium. Additionally, aliquots of each sample are cultivated on solid agar media to differentiate between turbidity caused by microbial growth and turbidity caused by sample reactions. Results are documented as Minimal Inhibitory Concentration (MIC) in ppm, i.e. the lowest concentration of the present polyimidazolium compound, which does not allow any microbial growth. The results are compiled in the below table 2.

TABLE 2
Minimal Inhibitory Concentration (MIC)

| | MIC [ppm] | | | | |
|---|---|---|---|---|---|
| | | | P. | C. | A. |
| Polymer | S. aureus | E. coli | aeruginosa | albicans | brasiliensis |
| Sample | ATCC 6538 | DSM 682 | ATCC 15442 | DSM 1386 | DSM 1988 |
| A | 0.98 | 1.95 | 0.98 | 7.81 | 15.63 |
| B | 0.98 | 1.95 | 0.98 | 7.81 | 15.63 |
| C | 7.81 | 3.91 | 3.91 | 15.63 | >15.63 |
| D | 0.98 | 0.98 | 0.98 | 15.63 | 15.63 |

### Evaluation of Bactericidal Activity in Solution:

The polymer of the invention (PIMS) is dissolved in water and tested according to European Standards (i.e. DIN EN 1040-2005 [in deionized water] and DIN EN 1276-2010 [in standardized hard water with additional soiling of 0.3% bovine albumin]). The final test composition contains the polymer of the invention in a concentration of 10 ppm (weight parts per million). Results are documented in table 3 as logarithmic reduction (Ig R) in comparison to the number of microorganism used for the test.

TABLE 3
Bactericidal activity in solution expressed as logarithmic reduction

| | [Ig R] | | [Ig R] | |
|---|---|---|---|---|
| | EN1040 (10 ppm, 5 min) | | EN1276 (10 ppm, 5 min) | |
| Polymer | S. aureus | P. aeruginosa | S. aureus | P. aeruginosa |
| Sample | ATCC 6538 | ATCC 15442 | ATCC 6538 | ATCC 15442 |
| A | 5.1 | >5.1 | 4.3 | 4.9 |
| B | >5.3 | >5.1 | 4.6 | 4.7 |
| C | 4.2 | >5.1 | 3.1 | 4.0 |
| D | >5.5 | >5.3 | 4.7 | 4.2 |

### Evaluation of Levurocidal Activity in Solution:

The polymer of the invention (PIMS) is dissolved in water and tested according to DIN EN 1275-2006 (in deionized water; final test composition containing the polymer of the invention in a concentration of 10 ppm). Results are documented in table 4 as logarithmic reduction (Ig R) in comparison to the number of microorganism used within the test.

TABLE 4
Levurocidal activity in solution expressed as logarithmic reduction

| | [lg R] |
|---|---|
| | EN1275 (10 ppm, 15 min) |
| | C. albicans |
| Polymer Sample | DSM 1386 |
| A | >4.4 |
| B | >4.4 |
| C | 3.2 |
| D | >4.4 |

### Evaluation of Bactericidal Activity in Formulation:

The polymer of the invention (PIMS) is dissolved in standardized hard water (DIN EN 1276-2010), then further components such as nonionic surfactant and chelating agent is added, finally pH is adjusted to 8.5. The complete formulation, representing an All-purpose-cleaner with a high amount of nonionic surfactant, is shown in table 5. For the purpose of comparison, a further formulation is prepared, whose composition is identical, except that the quantity of polymer of the invention is replaced by water.

TABLE 5
All-purpose-cleaner formulation

| | Test formulation |
|---|---|
| Ingredients | [%] (w/w) |
| Glucopon ® 425 N/HH | 5.0 |
| (Alkylpolyglucosid, 50%) | |
| Trilon M ® (MGDA) | 0.3 |
| Citric acid | 0.8 |
| NaOH | 0.5 |
| Sodium bicarbonate | 0.8 |
| 50% b.w. aq. Soln. of Polymer | 0.25 |
| of the invention | |
| Standardized hard Water | ad 10.0 |

Antimicrobial testing of the above formulation (pH 8) is done according to European Standard (DIN EN 1276-2010) under dirty conditions, i.e. additional soiling of 0.3% bovine albumin, as 80% (i.e. the 50% aq. solution of the test polymer is diluted according to standard to obtain a 0.125% formulation, which constitutes 80% of the final test composition thus containing 0.1% of the test polymer [all percentages per weight]). Results are documented in table 6 as logarithmic reduction (Ig R) in comparison to the number of microorganism grown in a reference formulation with the same composition but without biocide.

TABLE 6
Bactericidal activity in solution expressed as logarithmic reduction

| | [lg R] | |
|---|---|---|
| | EN1276 (0.1%, 5 min) | |
| | S. aureus | P. aeruginosa |
| Polymer Sample | ATCC 6538 | ATCC 15442 |
| B | >5.3 | >5.3 |
| None | <1.2 | <1.0 |

### Evaluation of Levurocidal Activity in Formulation:

A further formulation (All-purpose-cleaner) of composition as shown in table 7 is prepared and tested in analogy to the method described with relation to the above tables 5 and 6.

TABLE 7
All-purpose-cleaner formulation

| Ingredients | Test formulation [%] (w/w) |
|---|---|
| Glucopon ® 215 UP (C8-C10- | 0.78 |
| Alkylpolyglucoside) | |
| Trilon M ® (MGDA) | 0.6 |
| Lutensol ® XP 70 (C10-Guerbet | 0.5 |
| alcohol; 7-fold eth-oxylated) | |
| Triethanolamine | 0.5 |
| 50% b.w. aq. Soln. of Polymer | 0.15 |
| of the invention | |
| Standardized hard Water | ad 100 |

The cleaning formulation of Tab. 7 (pH 8.5) is tested in accordance with DIN EN 1650-2013 (standardized hard water containing 375 mg/i CaCO₃ and under dirty conditions [0.3% bovine albumin]), results are shown in the below table 8.

Levurocidal activity in solution expressed as logarithmic reduction

| | [lg R] |
|---|---|
| | EN1650 (0.06%, 15 min) |
| | C. albicans |
| Polymer Sample | ATCC 10231 |
| D | 3.6 |
| none | <1 |

In addition, the Antimicrobial All-purpose-cleaner formulation shown in table 7 is tested on hard surface according to European Standard EN13697-2001 using standardized hard water containing 375 mg/l CaCO₃ and under dirty conditions, i.e. additional soiling of 0.3% bovine albumin. Results of the bactericidal activity are documented in table 9 as the logarithmic microbicidal effect (ME) in comparison to the number of microorganism grown in a reference formulation with the same composition but without biocide.

TABLE 9
Bactericidal activity on hard surface
expressed as logarithmic microbicidal effect

| | Microbicidal effect (ME) |
|---|---|
| | EN13687 (0.075%, 5 min) |
| Polymer Sample | S. aureus ATCC 6538 |
| D | >7 |
| None | 0.2 |

### Evaluation of Antimicrobial Activity in Formulation—Material Protection:

A stock solution of the polymer of the invention is prepared in deionized water and mixed together with the filling material and the dispersant. The complete formulation, representing a composition of a mineral slurry typically used in paper manufacturing is shown in table 10. For the purpose of comparison, a further formulation is prepared, whose composition is identical, except that the quantity of polymer of the invention is replaced by water.

TABLE 10
Mineral slurry base formulation

| Ingredients | Test formulation [%] (w/w) |
|---|---|
| Precarb (Calcium carbonate) | 47.5 |
| Dispex ® AA 4140 | 0.475 |
| Polymer D (50%) | 0.01/0.002 |
| Deionized Water | ad 100 |

Antimicrobial testing of the above formulation is done according to European Standard DIN EN ISO 11930-2013. Results of antimicrobial activity are documented in table 11 and 12 as number of surviving colony forming units (cfu/ml) after certain periods of time in comparison to the number of microorganism grown in a reference formulation with the same composition but without biocide.

TABLE 11
Antibacterial activity in solution expressed as number of surviving cells

| | cfu/ml | | | | | |
|---|---|---|---|---|---|---|
| | S. aureus | | E. coli | | P. aeruginosa ATCC | |
| | ATCC 6538 | | ATCC 8739 | | 15442 | |
| | Inoculum t₀: | | Inoculum t₀: | | Inoculum t₀: | |
| | 5.0 × 10⁵/ml product | | 8.7 × 10⁵/ml product | | 5.2 × 10⁵/ml product | |
| | Reference | Polymer | Reference | Polymer | Reference | Polymer |
| | without | sample D | without | sample D | without | sample D |
| Contact time | biocide | (10 ppm) | biocide | (10 ppm) | biocide | (10 ppm) |
| 0 d | 1.5E+06 | — | 1.2E+06 | — | 7.0E+05 | — |
| 7 d | 1.3E+06 | <300 | 5.4E+06 | <300 | 7.4E+06 | <300 |
| 14 d | 3.3E+06 | <300 | 6.2E+06 | <300 | 1.9E+07 | <300 |
| 28 d | 3.4E+05 | <300 | 1.9E+05 | <300 | 7.3E+07 | <300 |

TABLE 12
Antifungal activity in solution
expressed as number of surviving cells

| | cfu/mL | | | |
|---|---|---|---|---|
| | C. albicans ATCC 10231 | | A. brasiliensis DSM 1988 | |
| | Inoculum t₀: | | Inoculum t₀: | |
| | 5.7 × 10⁴/ml product | | 4.9 × 10⁴/ml product | |
| | | Polymer | | Polymer |
| | Reference | sample D | Reference | sample D |
| Contact time | without biocide | (10 ppm) | without biocide | (50 ppm) |
| 0 d | 8.5E+04 | — | 1.7E+05 | — |
| 7 d | 4.9E+04 | <300 | 9.4E+04 | 1.4E+03 |
| 14 d | 5.8E+03 | <300 | 8.7E+04 | <150 |
| 28 d | 2.3E+05 | <300 | 8.7E+04 | <150 |

In a further test the polymer of invention is incorporated into a deodorant roll-on formulation as an example for a personal-care composition (see table 13). Therefore components of phase A and B are heated-up to 70° C. Under stirring phase A is incorporated into phase B and homogenized with a dispersing instrument. After cool down procedure the homogenized mixture is added to phase C at 30° C. After stirring pH adjustment is done to pH 5.5-6 and residual amounts of water are added.

TABLE 13
Deodorant roll-on formulation

| Phase | Ingredients | Test formulation [%] (w/w) |
|---|---|---|
| A | Paraffin | 8 |
| | Myritol ® 318 | 5 |
| | Cetiol ® CC | 3 |
| | Arlatone ® 983 P | 2 |
| | Brij ® 76 | 2 |
| | Lanette ® 16 | 0.5 |
| B | Deionized water | 67.6 |
| | Propylene Glycol | 2 |
| C | Polymer D (50%) | 0.2 |
| | Deionized water | 4.737 |
| D | Sodium Hydroxide | pH 5.5-6.0 |
| E | Deionized water | Add 100 |

Antimicrobial testing of the above formulation is done according to European Standard DIN EN ISO 11930-2013. Results of antimicrobial activity are documented in table 14 as number of surviving colony forming units (cfu/ml) after certain periods of time in comparison to the number of microorganism grown in a reference formulation with the same composition but without biocide.

TABLE 14
Antibacterial activity in deo-formulation expressed as number of surviving cells

| | cfu/ml | | | | | |
|---|---|---|---|---|---|---|
| | S. aureus | | | | P. aeruginosa ATCC | |
| | ATCC 6538 | | E. coli ATCC 8739 | | 15442 | |
| | Inoculum t₀: | | Inoculum t₀: | | Inoculum t₀: | |
| | 5.0 × 10⁵/ml product | | 8.7 × 10⁵/ml product | | 5.2 × 10⁵/ml product | |
| | Reference | Polymer | Reference | Polymer | Reference | Polymer |
| | without | sample D | without | sample D | without | sample D |
| Contact time | biocide | (0.1%) | biocide | (0.1%) | biocide | (0.1%) |
| 0 d | 8.9E+05 | — | 5.8E+05 | — | >1.0E+05 | — |
| 7 d | 1.5E+06 | <140 | 7.7E+06 | <140 | 2.4E+06 | <140 |
| 14 d | 1.1E+07 | <140 | 6.7E+06 | <140 | 5.2E+06 | <140 |
| 28 d | 8.2E+05 | <140 | 6.9E+05 | <140 | 1.3E+06 | <140 |

### Comparison of Antimicrobial Properties of Alkylated and Non-Alkylated Polymer:

### a) Determination of Bacterial Growth Inhibition in a Batch Culture

The polymer of the invention and a non-alkylated polymer are dissolved in Müller-Hinton Broth and inoculated with a bacterial solution resulting in around 107 cells per ml in the test approach respectively. Cultures inoculated with S. aureus (ATCC 6538) contain 229 nanomoles (nM) of the polymer, cultures inoculated with P. aeruginosa (ATCC 15442) contain 114 nanomoles (nM) of the polymer. Control experiments are done storing the cultures in water instead. Samples are stored at 37° C. under shaking (150/min). Aliquots are measured at certain time points with regard to optical density at 620 nm as shown in Tables 15 and 16. The results clearly show beneficial growth inhibition for the alkylated polymer.

TABLE 15
Inhibition of bacterial growth (S. aureus), OD(620 nm)

| | OD(620 nm) after storage for | | | |
|---|---|---|---|---|
| Polymer Sample | 2 h | 4 h | 6 h | 8 h |
| D | 0.003 | 0.003 | 0.004 | 0.010 |
| C10 | 0.004 | 0.163 | 0.583 | 1.274 |
| none | 0.016 | 0.240 | 0.663 | 1.330 |

TABLE 16
Inhibition of bacterial growth (P. aeruginosa), OD(620 nm)

| | OD(620 nm) after storage for | | | |
|---|---|---|---|---|
| Polymer Sample | 2 h | 4 h | 6 h | 8 h |
| D | 0.003 | 0.004 | 0.006 | 0.284 |
| C10 | 0.002 | 0.006 | 0.086 | 1.540 |
| none | 0.002 | 0.019 | 0.272 | 2.800 |

### b) Evaluation of Bactericidal Activity in Solution

The polymer of the invention and a non-alkylated polymer are dissolved in water and tested according to European Standard DIN EN 1276-2010 under dirty conditions, i.e. in standardized hard water with additional soiling of 0.3% bovine albumin. The final test composition contains the polymer in a concentration of 1.17 μM respectively. Results are documented in Table 17 as logarithmic reduction (Ig R) in comparison to the number of microorganism used for the test.

TABLE 17
Bactericidal activity in solution expressed as logarithmic reduction

| | [lg R] | | [lg R] | |
|---|---|---|---|---|
| | EN1276 (1.17 μM, 5 min) | | EN1276 (1.17 μM, 1 min) | |
| Polymer | S. aureus | P. aeruginosa | S. aureus | P. aeruginosa |
| Sample | ATCC 6538 | ATCC 15442 | ATCC 6538 | ATCC 15442 |
| D | 4.7 | >5.2 | 3.3 | >5.2 |
| C10 | 3.6 | >5.1 | 1.8 | 4.7 |

### Evaluation of Antibacterial Activity on Textiles Via Softener Application

Cotton fabrics are pre-washed with a commercial detergent and rinsed in autoclaved tap water before the softener formulation of composition as shown in Table 18 is applied at 0.15% for 20 minutes to the textile material under washing conditions again (a reference fabric without antibacterial finish is only prewashed and rinsed while another fabric is treated with a softener formulation without the polymer of invention).

Then the fabrics are tested regarding antibacterial activity according to AATCC-100-2012 which has been slightly modified with regard to sample size and inoculum volume. Moreover calculation of antibacterial activity (“R”) is done in comparison to reference fabric after incubation period and not after inoculation as prescribed in the method. Results are documented in Table 19 as logarithmic reduction (Ig R) in comparison to the number of microorganism grown on the reference fabric without antimicrobial finishing.

TABLE 18
Softener formulation

| Ingredients | Test Formulation [% w/w] |
|---|---|
| Dehyquart ® AU-57 | 18 |
| Lutensol ® AT 25 | 1 |
| Sodium chloride | 0.5 |
| 50% b.w. aq. Soln. of | 5.0 |
| Polymer of the | |
| invention | |
| MSA | pH 3 |
| Water | ad 100 |

TABLE 19
Bactericidal activity on cotton fabrics expressed as
logarithmic reduction

| | lg R, 24 h | lg R, 24 h |
|---|---|---|
| Textile | S. aureus | K. pneumoniae |
| treatment | ATCC 6538 | DSM 789 |
| Softener | >5.8 | >6.5 |
| containing | | |
| polymer of | | |
| invention | | |
| Softener with- | <1 | <1 |
| out polymer of | | |
| invention | | |

Evaluation of Disinfectant Activity Against Pseudomonas aeruginosa Biofilm

The polymer of the invention is dissolved in water and tested according to American Standard ASTM E 2799-12 “Testing Disinfectant Efficacy against Pseudomonas aeruginosa Biofilm using the MBEC Assay” (in this test Pseudomonas aeruginosa biofilms are formed on polystyrene pegs and treated with biocidal solutions). Results are documented in Table 20 as logarithmic reduction (Ig R) of cfu/mm² in comparison to an untreated water control.

TABLE 20
Antibiofilm activity expressed as logarithmic reduction

| | [lg R] |
|---|---|
| | ASTM 2799 (80 ppm, 10 min) |
| Polymer Sample | P. aeruginosa ATCC 15442 |
| D | >4.4 |

1.-15. (canceled)

16. An antimicrobial composition comprising as an antimicrobial agent at least one polymeric, ionic compound comprising at least 5 imidazolium groups, which is obtainable by one of the methods (i) to (iii), and at least one carrier and/or at least one auxiliary agent, wherein
method (i) comprises reacting glyoxal, formaldehyde, at least one C₄-C₈alkylene diamine, at least one protic acid, and a C₈-C₂₀alkylamine,
method (ii) comprises a first step of reacting glyoxal, formaldehyde, at least one C₄-C₈alkylene diamine and at least one protic acid, followed by a second step of reacting the product obtained in the first step with a C₈-C₂₀alkyl compound,
method (iii) comprises a first step of reacting glyoxal, formaldehyde, at least one C₄-C₈alkylene diamine, at least one protic acid and a C₈-C₂₀alkylamine, followed by a second step of reacting the product obtained in the first step with a C₈-C₂₀alkyl compound.

17. The composition of claim 16, wherein, in the methods (i) to (iii), the C₄-C₈alkylene diamine is hexylene diamine, and/or the C₈-C₂₀alkylamine is dodecylamine.

18. The composition of claim 16, wherein, in the methods (i) to (iii), the protic acid is selected from the group consisting of C₁-C₂₀carboxylic acids and mineralic acids.

19. The composition according to claim 16, wherein the polymeric, ionic compound comprising at least 5 imidazolium groups obtainable by one of the methods (i) to (iii) comprises a polymer of the formula I′ 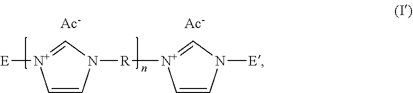
wherein
E is C₈-C₂₀ alkyl;
R is C₄-C₈alkylene;
Ac— is the anion of the protonic acid used according to methods (i) to (iii) and the index n ranges from 4 to 10000.

20. The composition according to claim 16, wherein the polymeric, ionic compound comprising at least 5 imidazolium groups obtainable by one of the methods (i) to (iii) comprises a polymer of the formula I, 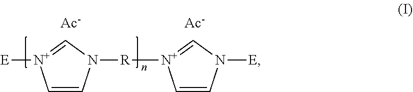
wherein E is dodecyl, R is hexylene, and Ac⁻ stands for the acetyl anion.

21. The composition according to claim 16, wherein the carrier and/or auxiliary agent comprises water and a surfactant.

22. The composition according to claim 16, wherein the carrier comprises an alcohol.

23. The composition according to claim 21, wherein the surfactant is selected from cationic, nonionic and amphoteric surfactants, block polymers, polyelectrolytes, and mixtures thereof.

24. The composition as claimed in claim 16, wherein the composition is
a plant protection composition; or
a personal care composition, or
a home care composition, or
a composition used for industrial or institutional or hospital disinfection,
a material protection composition, or
a pharmaceutical composition.

25. The composition according to claim 16, additionally containing a further antimicrobial agent which is a chloro benzene derivative of the formula (II) 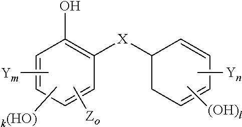
wherein
X is O, S or —CH2-
Y is Cl or Br,
Z is SO2H, NO2 or C1 to C4 alkyl,
k is 0 or 1,
l is 0 or 1,
m is 0, 1, 2, or 3,
n is 0, 1, 2, or 3, and
o is 0 or 1.

26. The composition as claimed in claim 16 further containing a tackifier or binder selected from the group consisting of polyvinylpyrrolidones; polyvinylacetates; polyvinyl alcohols; polyacrylates; biological or synthetic waxes; and cellulose ethers.

27. The composition of claim 24, which is a personal care composition further comprising an ingredient selected from the group consisting of abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents, skin protectants, solvents, foam boosters, hydrotropes, solubilizing agents, suspending agents, sunscreen agents, ultraviolet light absorbers, viscosity increasing agents, sequestrants, and keratolytics.

28. The composition of claim 31, which is a home care composition further comprising an additional component selected from the group consisting of enzymes, bleaches, whiteners, color care agents, fabric softeners, suds suppressors, dispersants, dye transfer inhibitors, chelating agents, aerosol propellants, gelling agents, thickening agents, and a combination thereof.

29. The composition as claimed in claim 16 containing the polymeric, ionic compound comprising at least 5 imidazolium groups in an amount ranging from 0.0001 to 30% by weight of the composition.

30. A method for combating harmful organisms or for protecting human beings, animals, materials or processes from the effects of these harmful organisms, wherein the habitat of the harmful organism or the human being, animal or material to be protected is brought into contact with a biocide composition or the biocide composition is employed in said process, wherein the biocide composition comprises at least one polymeric, ionic compound comprising imidazolium groups, where the biocide composition is as defined in claim 16; where the method is not a method for treatment of the human or animal body by surgery or therapy or diagnostic method practised on the human or animal body.

31. A polymeric, ionic compound comprising at least 5 imidazolium groups, which compound is obtainable by simultaneously reacting glyoxal, formaldehyde, at least one C₄-C₈alkylene diamine, at least one protic acid, and a C₈-C₂₀alkylamine.

32. The polymeric, ionic compound of claim 31, obtainable by simultaneously reacting 0.9-1.1 molar parts of glyoxal with 1 molar part of formaldehyde, 0.9-1.0 molar parts of the alkylene diamine, 0.01-0.2 molar parts of the alkylamine, and about 1.1 to 5 molar parts of the protic acid.

33. The polymeric, ionic compound of claim 31, which conforms to the formula I′ 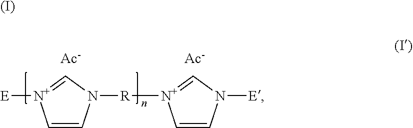
wherein
E is C₈-C₂₀alkyl;
R is C₄-C₈alkylene;
Ac— is the anion of a protonic acid; and
the index n ranges from 4 to 10000.

34. A process for the preparation of a polymeric, ionic compound comprising at least 5 imidazolium groups as described in claim 31, which process comprises simultaneously reacting glyoxal, formaldehyde, at least one C₄-C₈alkylene diamine, at least one protic acid, and a C₈-C₂₀alkylamine.

35. The process of claim 34, wherein simultaneously 0.9-1.1 molar parts of glyoxal are reacted with 1 molar part of formaldehyde, 0.9-1.0 molar parts of the alkylene diamine, 0.01-0.2 molar parts of the alkylamine, and about 1.1 to 5 molar parts of the protic acid.
